SEQUENCE LISTING (134 sequences)

SEQ ID NO: 1 - protein, 457 aa, Homo sapiens
QVQLVESGGGLVRPGGSLRLSCAASGFTFSDYFMTWIRQAPGKGLEWVSYINSIGSVIFYADSVEGRFTVSRDNTMKSLFLQMNSLTAEDTAVYYCARSCRISYGASGGFSCGMDVWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 127 aa, Homo sapiens
QVQLVESGGGLVRPGGSLRLSCAASGFTFSDYFMTWIRQAPGKGLEWVSYINSIGSVIFYADSVEGRFTVSRDNTMKSLFLQMNSLTAEDTAVYYCARSCRISYGASGGFSCGMDVWGQGTPVTVSS

SEQ ID NO: 3 - protein, 5 aa, Homo sapiens
DYFMT

SEQ ID NO: 4 - protein, 17 aa, Homo sapiens
YINSIGSVIFYADSVEG

SEQ ID NO: 5 - protein, 18 aa, Homo sapiens
SCRISYGASGGFSCGMDV

SEQ ID NO: 6 - protein, 213 aa, Homo sapiens
DIQMTQSPSSLSASVGDRVTITCQASQSIRENINWYQQKTGKAPKLLIYDASILETGVPSRFSGSGSGTHFTFTISSLQPEDMATYYCQQYHHLITFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 106 aa, Homo sapiens
DIQMTQSPSSLSASVGDRVTITCQASQSIRENINWYQQKTGKAPKLLIYDASILETGVPSRFSGSGSGTHFTFTISSLQPEDMATYYCQQYHHLITFGQGTRLDIK

SEQ ID NO: 8 - protein, 11 aa, Homo sapiens
QASQSIRENIN

SEQ ID NO: 9 - protein, 7 aa, Homo sapiens
DASILET

SEQ ID NO: 10 - protein, 8 aa, Homo sapiens
QQYHHLIT

SEQ ID NO: 11 - protein, 448 aa, Homo sapiens
QVQLVQSGAEVKKPGASVKVSCKASGYTFSVYGISWVRQAPGHGLEWMGWISAYNGDRGYAQKVQGRITMTTDTSASTAYMELRSLTSDDTAVYYCATTSSGVPGIYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 118 aa, Homo sapiens
QVQLVQSGAEVKKPGASVKVSCKASGYTFSVYGISWVRQAPGHGLEWMGWISAYNGDRGYAQKVQGRITMTTDTSASTAYMELRSLTSDDTAVYYCATTSSGVPGIYWGQGSLVTVSS

SEQ ID NO: 13 - protein, 5 aa, Homo sapiens
VYGIS

SEQ ID NO: 14 - protein, 17 aa, Homo sapiens
WISAYNGDRGYAQKVQG

SEQ ID NO: 15 - protein, 9 aa, Homo sapiens
TSSGVPGIY

SEQ ID NO: 16 - protein, 217 aa, Homo sapiens
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDNLSGPWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - protein, 111 aa, Homo sapiens
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDNLSGPWVFGGGTKLTVL

SEQ ID NO: 18 - protein, 13 aa, Homo sapiens
SGSSSNIGSNYVY

SEQ ID NO: 19 - protein, 7 aa, Homo sapiens
RNNQRPS

SEQ ID NO: 20 - protein, 12 aa, Homo sapiens
AAWDDNLSGPWV

SEQ ID NO: 21 - protein, 449 aa, Homo sapiens
QVQLQESGPGLVKPSETLSLTCTVSGGSISRDYWSFIRQPAGKGLEWIGRIYHNGDTNYNPSLKSRVTMSQDTSKNQFFLRLRHVTAADTAVYYCAREGSAGTFGQADWGQGTLVIVYSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 119 aa, Homo sapiens
QVQLQESGPGLVKPSETLSLTCTVSGGSISRDYWSFIRQPAGKGLEWIGRIYHNGDTNYNPSLKSRVTMSQDTSKNQFFLRLRHVTAADTAVYYCAREGSAGTFGQADWGQGTLVIVYS

SEQ ID NO: 23 - protein, 5 aa, Homo sapiens
RDYWS

SEQ ID NO: 24 - protein, 16 aa, Homo sapiens
RIYHNGDTNYNPSLKS

SEQ ID NO: 25 - protein, 11 aa, Homo sapiens
EGSAGTFGQAD

SEQ ID NO: 26 - protein, 215 aa, Homo sapiens
EIVMTQSPATLSVSPGERATLSCRASQSISNNLAWYQQKPGQAPRLLIYGASTRATGIPARFSGGGSGTEFTLTISSLQSEDFAVYYCQQYENWLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 108 aa, Homo sapiens
EIVMTQSPATLSVSPGERATLSCRASQSISNNLAWYQQKPGQAPRLLIYGASTRATGIPARFSGGGSGTEFTLTISSLQSEDFAVYYCQQYENWLLYTFGQGTKLEIK

SEQ ID NO: 28 - protein, 11 aa, Homo sapiens
RASQSISNNLA

SEQ ID NO: 29 - protein, 7 aa, Homo sapiens
GASTRAT

SEQ ID NO: 30 - protein, 10 aa, Homo sapiens
QQYENWLLYT

SEQ ID NO: 31 - protein, 453 aa, Homo sapiens
EVQLVESGGGLVKPGGSLRLSCAASGFTFSSYSMNWVRQAPGKGLEWVSSISSSSSYIYYADSLKGRFTVSRDNAKKSLYLQMNSLRAEDTAVYYCARVDYGTNSEVGRFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 123 aa, Homo sapiens
EVQLVESGGGLVKPGGSLRLSCAASGFTFSSYSMNWVRQAPGKGLEWVSSISSSSSYIYYADSLKGRFTVSRDNAKKSLYLQMNSLRAEDTAVYYCARVDYGTNSEVGRFDPWGQGTLVTVSS

SEQ ID NO: 33 - protein, 5 aa, Homo sapiens
SYSMN

SEQ ID NO: 34 - protein, 17 aa, Homo sapiens
SISSSSSYIYYADSLKG

SEQ ID NO: 35 - protein, 14 aa, Homo sapiens
VDYGTNSEVGRFDP

SEQ ID NO: 36 - protein, 217 aa, Homo sapiens
QSVLTQPPSVSGTPGQRVTISCTGSSSNIGAGYAVHWYQQLPGTAPKLLIYDSINRPSGVPDRFSGSKSGTSASLAITGLQTEDEADYYCHSYDRSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 37 - protein, 111 aa, Homo sapiens
QSVLTQPPSVSGTPGQRVTISCTGSSSNIGAGYAVHWYQQLPGTAPKLLIYDSINRPSGVPDRFSGSKSGTSASLAITGLQTEDEADYYCHSYDRSLSGSVFGGGTKLTVL

SEQ ID NO: 38 - protein, 14 aa, Homo sapiens
TGSSSNIGAGYAVH

SEQ ID NO: 39 - protein, 7 aa, Homo sapiens
DSINRPS

SEQ ID NO: 40 - protein, 11 aa, Homo sapiens
HSYDRSLSGSV

SEQ ID NO: 41 - protein, 453 aa, Homo sapiens
QVQLVQSGTELKKPGASVKVSCKASGYTFTDYSMHWLRQAPGESLEWMGWVNGGPLDTKYSPTFQDRVSFSRDVFATTAYMDLSSLGPEDTAVYYCARGWGITAYRGDAFDLWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 123 aa, Homo sapiens
QVQLVQSGTELKKPGASVKVSCKASGYTFTDYSMHWLRQAPGESLEWMGWVNGGPLDTKYSPTFQDRVSFSRDVFATTAYMDLSSLGPEDTAVYYCARGWGITAYRGDAFDLWGQGTMVIVSS

SEQ ID NO: 43 - protein, 5 aa, Homo sapiens
DYSMH

SEQ ID NO: 44 - protein, 17 aa, Homo sapiens
WVNGGPLDTKYSPTFQD

SEQ ID NO: 45 - protein, 14 aa, Homo sapiens
GWGITAYRGDAFDL

SEQ ID NO: 46 - protein, 214 aa, Homo sapiens
SFVLTQPPSVSVAPGKSARITCGASNIESKSVHWYQQKPGQAPVVVVYDDDDRPSGIPERFSGSNSGNTATLIINRVEVGDEADYYCQVWDSTSDMYVFGAGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 47 - protein, 108 aa, Homo sapiens
SFVLTQPPSVSVAPGKSARITCGASNIESKSVHWYQQKPGQAPVVVVYDDDDRPSGIPERFSGSNSGNTATLIINRVEVGDEADYYCQVWDSTSDMYVFGAGTKVTVL

SEQ ID NO: 48 - protein, 11 aa, Homo sapiens
GASNIESKSVH

SEQ ID NO: 49 - protein, 7 aa, Homo sapiens
DDDDRPS

SEQ ID NO: 50 - protein, 11 aa, Homo sapiens
QVWDSTSDMYV

SEQ ID NO: 51 - protein, 450 aa, Homo sapiens
QVQLVESGGGVAQPGRSLRLSCAASGFTLSNYGMHWVRQAPGKGLEWVAVIWYDGRNKYYADSVKGRFTISRDDFKNTLDLQMNSLRAEDTAVYYCARDQHAGITMIEDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 120 aa, Homo sapiens
QVQLVESGGGVAQPGRSLRLSCAASGFTLSNYGMHWVRQAPGKGLEWVAVIWYDGRNKYYADSVKGRFTISRDDFKNTLDLQMNSLRAEDTAVYYCARDQHAGITMIEDWGQGTLVTVSS

SEQ ID NO: 53 - protein, 5 aa, Homo sapiens
NYGMH

SEQ ID NO: 54 - protein, 17 aa, Homo sapiens
VIWYDGRNKYYADSVKG

SEQ ID NO: 55 - protein, 11 aa, Homo sapiens
DQHAGITMIED

SEQ ID NO: 56 - protein, 216 aa, Homo sapiens
QSALTQPPSASGSPGQSVTISCTGTSSDVGYFKYVSWYQQHPGKAPKLLIYEVSKRPSGVPDRFSGSRSGNTASLTVSGLQADDEADYYCGSYAGNNNIIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 57 - protein, 110 aa, Homo sapiens
QSALTQPPSASGSPGQSVTISCTGTSSDVGYFKYVSWYQQHPGKAPKLLIYEVSKRPSGVPDRFSGSRSGNTASLTVSGLQADDEADYYCGSYAGNNNIIFGGGTKLTVL

SEQ ID NO: 58 - protein, 14 aa, Homo sapiens
TGTSSDVGYFKYVS

SEQ ID NO: 59 - protein, 7 aa, Homo sapiens
EVSKRPS

SEQ ID NO: 60 - protein, 10 aa, Homo sapiens
GSYAGNNNII

SEQ ID NO: 61 - protein, 449 aa, Homo sapiens
QEQLVESGGDLVKPGGSLRITCAASGFTLSGYALVWVRQAPGRGLEWIADITASSTYINYAASVKGRFTISRDNAQSSLFLQMSSLRADDTGVYFCARLPHLPGFFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 119 aa, Homo sapiens
QEQLVESGGDLVKPGGSLRITCAASGFTLSGYALVWVRQAPGRGLEWIADITASSTYINYAASVKGRFTISRDNAQSSLFLQMSSLRADDTGVYFCARLPHLPGFFDLWGRGTLVSVSS

SEQ ID NO: 63 - protein, 5 aa, Homo sapiens
GYALV

SEQ ID NO: 64 - protein, 17 aa, Homo sapiens
DITASSTYINYAASVKG

SEQ ID NO: 65 - protein, 10 aa, Homo sapiens
LPHLPGFFDL

SEQ ID NO: 66 - protein, 217 aa, Homo sapiens
QSVLTQPPSVSGAPGQRVTISCTGNSSNIGAGYDVNWYQHLPGTAPKLLISVNTNRPSGVPDRFSGSMSGTSASLAITGLQAEDEADYYCQSYDRRLSGYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 67 - protein, 111 aa, Homo sapiens
QSVLTQPPSVSGAPGQRVTISCTGNSSNIGAGYDVNWYQHLPGTAPKLLISVNTNRPSGVPDRFSGSMSGTSASLAITGLQAEDEADYYCQSYDRRLSGYVFGTGTKVTVL

SEQ ID NO: 68 - protein, 14 aa, Homo sapiens
TGNSSNIGAGYDVN

SEQ ID NO: 69 - protein, 7 aa, Homo sapiens
VNTNRPS

SEQ ID NO: 70 - protein, 11 aa, Homo sapiens
QSYDRRLSGYV

SEQ ID NO: 71 - protein, 455 aa, Homo sapiens
QVQLVQSGAEMKKPGASVKVSCKASGYTFTDYFIHWVRQAPGQGLEWMGWVNPNSGATNYAQKFQGRVTMTGDTSLSAAYVDLSRLSSDDSAVYYCARDRGLGISSTRNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 125 aa, Homo sapiens
QVQLVQSGAEMKKPGASVKVSCKASGYTFTDYFIHWVRQAPGQGLEWMGWVNPNSGATNYAQKFQGRVTMTGDTSLSAAYVDLSRLSSDDSAVYYCARDRGLGISSTRNDAFDIWGQGTTVTVSS

SEQ ID NO: 73 - protein, 5 aa, Homo sapiens
DYFIH

SEQ ID NO: 74 - protein, 17 aa, Homo sapiens
WVNPNSGATNYAQKFQG

SEQ ID NO: 75 - protein, 16 aa, Homo sapiens
DRGLGISSTRNDAFDI

SEQ ID NO: 76 - protein, 214 aa, Homo sapiens
DIQLTQSPSSLSASVGDRVTITCQPSQGIRNFLNWYQQKPGKAPNLLIYDASNLETGVASRFSGSGSGTHFTLTISSLQPEDIATYFCQQYHSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 107 aa, Homo sapiens
DIQLTQSPSSLSASVGDRVTITCQPSQGIRNFLNWYQQKPGKAPNLLIYDASNLETGVASRFSGSGSGTHFTLTISSLQPEDIATYFCQQYHSLPLTFGGGTKVEIK

SEQ ID NO: 78 - protein, 11 aa, Homo sapiens
QPSQGIRNFLN

SEQ ID NO: 79 - protein, 7 aa, Homo sapiens
DASNLET

SEQ ID NO: 80 - protein, 9 aa, Homo sapiens
QQYHSLPLT

SEQ ID NO: 81 - protein, 451 aa, Homo sapiens
QVQLVQSGAEVKKPGASVKVSCKASGYTFADYYIHWLRQAPGQGLEWLGRINPSNSRTTYPQKFQGRVTVTRDTSTSTVYMELSSLRSADTAVYYCARDGLRMGYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 121 aa, Homo sapiens
QVQLVQSGAEVKKPGASVKVSCKASGYTFADYYIHWLRQAPGQGLEWLGRINPSNSRTTYPQKFQGRVTVTRDTSTSTVYMELSSLRSADTAVYYCARDGLRMGYYYMDVWGKGTTVTVSS

SEQ ID NO: 83 - protein, 5 aa, Homo sapiens
DYYIH

SEQ ID NO: 84 - protein, 17 aa, Homo sapiens
RINPSNSRTTYPQKFQG

SEQ ID NO: 85 - protein, 12 aa, Homo sapiens
DGLRMGYYYMDV

SEQ ID NO: 86 - protein, 217 aa, Homo sapiens
QSVLTQPPSVSGAPGQRVTISCTGSSSNIGAGYDVHWYQQIPGTAPKLLIFGNYNRPSGVPDRFSASKSGTSASLAITGLQAEDEAHYYCQSYDTTLDGAVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 87 - protein, 111 aa, Homo sapiens
QSVLTQPPSVSGAPGQRVTISCTGSSSNIGAGYDVHWYQQIPGTAPKLLIFGNYNRPSGVPDRFSASKSGTSASLAITGLQAEDEAHYYCQSYDTTLDGAVFGGGTKLTVL

SEQ ID NO: 88 - protein, 14 aa, Homo sapiens
TGSSSNIGAGYDVH

SEQ ID NO: 89 - protein, 7 aa, Homo sapiens
GNYNRPS

SEQ ID NO: 90 - protein, 11 aa, Homo sapiens
QSYDTTLDGAV

SEQ ID NO: 91 - protein, 454 aa, Homo sapiens
QVQLVQSGAEVKKPGSSVRVSCKASGGTFSTYGINWVRQARGQGLEWMGTVIPTFEKTYYAQGFQGRVTIAADGSKNTVYMELRRLTSEDSAIYFCTRFFTPYCSGGSCYLDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 124 aa, Homo sapiens
QVQLVQSGAEVKKPGSSVRVSCKASGGTFSTYGINWVRQARGQGLEWMGTVIPTFEKTYYAQGFQGRVTIAADGSKNTVYMELRRLTSEDSAIYFCTRFFTPYCSGGSCYLDLWGPGTLVTVSS

SEQ ID NO: 93 - protein, 5 aa, Homo sapiens
TYGIN

SEQ ID NO: 94 - protein, 17 aa, Homo sapiens
TVIPTFEKTYYAQGFQG

SEQ ID NO: 95 - protein, 15 aa, Homo sapiens
FFTPYCSGGSCYLDL

SEQ ID NO: 96 - protein, 214 aa, Homo sapiens
QSVLTQPPSVSGAPGQSVTISCAGSSSNIGAGYDVHWYQHLPGTAPRLLIYSNTNRPSGVPDRFSGSKSGASASLAITGLQAEDEADYYCQSYDSGVVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - protein, 108 aa, Homo sapiens
QSVLTQPPSVSGAPGQSVTISCAGSSSNIGAGYDVHWYQHLPGTAPRLLIYSNTNRPSGVPDRFSGSKSGASASLAITGLQAEDEADYYCQSYDSGVVFGGGTKLTVL

SEQ ID NO: 98 - protein, 14 aa, Homo sapiens
AGSSSNIGAGYDVH

SEQ ID NO: 99 - protein, 7 aa, Homo sapiens
SNTNRPS

SEQ ID NO: 100 - protein, 8 aa, Homo sapiens
QSYDSGVV

SEQ ID NO: 101 - protein, 452 aa, Homo sapiens
QVQLVQSGAEVQKPGSSVKVSCKTSGDTFNNYALSWVRQAPGQGPEWMGRIIPLVEMANYAQKFQGRVTITADKSTTTIYMELSSLRSEDTAMYYCARDGYQGGFPYFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 122 aa, Homo sapiens
QVQLVQSGAEVQKPGSSVKVSCKTSGDTFNNYALSWVRQAPGQGPEWMGRIIPLVEMANYAQKFQGRVTITADKSTTTIYMELSSLRSEDTAMYYCARDGYQGGFPYFFDSWGQGTLVTVSS

SEQ ID NO: 103 - protein, 5 aa, Homo sapiens
NYALS

SEQ ID NO: 104 - protein, 17 aa, Homo sapiens
RIIPLVEMANYAQKFQG

SEQ ID NO: 105 - protein, 13 aa, Homo sapiens
DGYQGGFPYFFDS

SEQ ID NO: 106 - protein, 214 aa, Homo sapiens
EVVLTQSPGTLSLSPGERATLSCRASQSVSNYLAWYQQKPGQAPRLLISAASSRATGIPDRFGGSGSGADFTLTISRLEPEDFAVYYCQQYGFSPGTFGQGTKVEIRRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 107 - protein, 107 aa, Homo sapiens
EVVLTQSPGTLSLSPGERATLSCRASQSVSNYLAWYQQKPGQAPRLLISAASSRATGIPDRFGGSGSGADFTLTISRLEPEDFAVYYCQQYGFSPGTFGQGTKVEIR

SEQ ID NO: 108 - protein, 11 aa, Homo sapiens
RASQSVSNYLA

SEQ ID NO: 109 - protein, 7 aa, Homo sapiens
AASSRAT

SEQ ID NO: 110 - protein, 9 aa, Homo sapiens
QQYGFSPGT

SEQ ID NO: 111 - protein, 448 aa, Homo sapiens; identical to SEQ ID NO: 11

SEQ ID NO: 112 - protein, 118 aa, Homo sapiens; identical to SEQ ID NO: 12

SEQ ID NO: 113 - protein, 5 aa, Homo sapiens
VYGIS

SEQ ID NO: 114 - protein, 17 aa, Homo sapiens
WISAYNGDRGYAQKVQG

SEQ ID NO: 115 - protein, 9 aa, Homo sapiens
TSSGVPGIY

SEQ ID NO: 116 - protein, 214 aa, Homo sapiens
SYALVQPPSVSVSPGQTARITCFGPALPKKYAYWYQHKSGQAPMLVIYEDTKRPSGIPQRFSGSTSGATATLTIRGAQVEDEADYYCYSTDSSGHERVFGGGTKLTVLGQPKAAPSVTLFPPSSEELQANKATLVCLISDFYPGAVTVAWKADSSPVKAGVETTAPSKQSNNKYAASSYLSLTPEQWKSHKSYSCQVTHEGSTVEKTVAPTECS

SEQ ID NO: 117 - protein, 108 aa, Homo sapiens
SYALVQPPSVSVSPGQTARITCFGPALPKKYAYWYQHKSGQAPMLVIYEDTKRPSGIPQRFSGSTSGATATLTIRGAQVEDEADYYCYSTDSSGHERVFGGGTKLTVL

SEQ ID NO: 118 - protein, 11 aa, Homo sapiens
FGPALPKKYAY

SEQ ID NO: 119 - protein, 7 aa, Homo sapiens
EDTKRPS

SEQ ID NO: 120 - protein, 11 aa, Homo sapiens
YSTDSSGHERV

SEQ ID NO: 121 - protein, 455 aa, Homo sapiens
RVQLVQSGAEVKKPGSSVRVSCKASGGTFSNYSISWVRQVPGQGLEWMGRIIPILPLSSYPQQFQGRVAITADKSSTTAYMELSSLRIEDTAIYYCARDALRPEGGYYYYYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 125 aa, Homo sapiens
RVQLVQSGAEVKKPGSSVRVSCKASGGTFSNYSISWVRQVPGQGLEWMGRIIPILPLSSYPQQFQGRVAITADKSSTTAYMELSSLRIEDTAIYYCARDALRPEGGYYYYYFDVWGTGTTVTVSS

SEQ ID NO: 123 - protein, 5 aa, Homo sapiens
NYSIS

SEQ ID NO: 124 - protein, 17 aa, Homo sapiens
RIIPILPLSSYPQQFQG

SEQ ID NO: 125 - protein, 16 aa, Homo sapiens
DALRPEGGYYYYYFDV

SEQ ID NO: 126 - protein, 214 aa, Homo sapiens
EIELTQSPGTLSLSPGERATLSCRANQIIGDTSLAWYQQKPGQAPRLLIFGASSRATGIPDRFSGSGFGSDFTLTITSLEPEDFAVYYCQQYGSSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 107 aa, Homo sapiens
EIELTQSPGTLSLSPGERATLSCRANQIIGDTSLAWYQQKPGQAPRLLIFGASSRATGIPDRFSGSGFGSDFTLTITSLEPEDFAVYYCQQYGSSPTFGQGTKVEIK

SEQ ID NO: 128 - protein, 12 aa, Homo sapiens
RANQIIGDTSLA

SEQ ID NO: 129 - protein, 7 aa, Homo sapiens
GASSRAT

SEQ ID NO: 130 - protein, 8 aa, Homo sapiens
QQYGSSPT

SEQ ID NO: 131 - DNA, 1620 bp, Human Metaneumovirus
atgtcttggaaagtgatgatcatcatttcgttactcataacaccccagcacgggctaaaggagagttatttggaagaatcatgcagtactataactgagggatacctcagtgttttaagaacaggctggtacactaatgtcttcacattagaagttggtgatgttgaaaatcttacatgtactgatggacctagcttaatcaaaacagaacttgacctaacaaaaagtgctttaagggaactcaaaacagtctctgctgatcagttagctagagaggagcaaattgaaaatcccagacaatcaagatttgtcctaggtgcaatagctctcggagttgctacagcagcagcagtcacagcaggcattgcaatagccaaaaccataaggcttgagagtgaggtgaatgcaattaaaggtgctctcaaacaaactaatgaagcagtatccacattaggaaatggtgtgcgggtcctagccactgcagtgagagagctaaaagaatttgtgagcaaaaatctgactagtgcaatcaacaggaacaaatgtgacattgctgatctgaagatggctgtcagcttcagtcaattcaacagaagatttctaaatgttgtgcggcagttttcagacaatgcagggataacaccagcaatatcattggacctaatgactgatgctgaattggccagagctgtatcatacatgccaacatctgcaggacagataaaactgatgttggagaaccgcgcaatggtaaggagaaaaggatttggaatcctaataggggtctacggaagctctgtgatttacatggttcaattgccgatctttggtgtcatagatacaccttgttggataatcaaggcagctccctcttgctcagaaaaaaacgggaattatgcttgcctcctaagagaggatcaagggtggtattgtaaaaatgcaggatccactgtttactacccaaatgaaaaagactgtgaaacaagaggtgatcatgttttttgtgacacagcagcagggatcaatgttgctgagcaatcaagagaatgcaacatcaacatatctactaccaactacccatgcaaagtcagcacaggaagacaccctataagcatggttgcactatcacctctcggtgctttggtggcttgctataaaggggtaagctgctcgattggcagcaatcgagttggaatcatcaaacaattacctaaaggctgctcatacataactaaccaggatgcagacactgtaacaattgacaataccgtgtatcaactaagcaaagttgaaggtgaacagcatgtaataaaagggagaccagtttcaagcagttttgatccaatcaggtttcctgaggatcagttcaatgttgcgcttgatcaagtcttcgaaagcattgagaacagtcaggcactggtggaacagtcaaacaaaattctaaacagtgcagaaaaaggaaacactggtttcattattgtaataattttggttgctgttcttggtttaaccatgatttcagtgagcatcatcatcataatcaagaaaacaaggaagcccacaggagcacctccagagctgaatggtgtcaccaacggcggtttcataccacatagttag

SEQ ID NO: 132 - protein, 539 aa, Artificial Sequence
  Fusion Protein Sequence
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIRFPEDQFNVALDQVFESIENSQALVEQSNKILNSAEKGNTGFIIVIILVAVLGLTMISVSIIIIIKKTRKPTGAPPELNGVTNGGFIPHS

SEQ ID NO: 133 - DNA, 1738 bp, Artificial Sequence
  HA-V5-Full
aagcttgccaccatgtcttggaaagtgatgatcatcatttcgttactcataacaccccagcacgggaccatgtacccatacgatgttccagattacgctctaaaggagagttatttggaagaatcatgcagtactataactgagggatacctcagtgttttaagaacaggctggtacactaatgtcttcacattagaagttggtgatgttgaaaatcttacatgtactgatggacctagcttaatcaaaacagaacttgacctaacaaaaagtgctttaagggaactcaaaacagtctctgctgatcagttagctagagaggagcaaattgaaaatcccagacaatcaagatttgtcctaggtgcaatagctctcggagttgctacagcagcagcagtcacagcaggcattgcaatagccaaaaccataaggcttgagagtgaggtgaatgcaattaaaggtgctctcaaacaaactaatgaagcagtatccacattaggaaatggtgtgcgggtcctagccactgcagtgagagagctaaaagaatttgtgagcaaaaatctgactagtgcaatcaacaggaacaaatgtgacattgctgatctgaagatggctgtcagcttcagtcaattcaacagaagatttctaaatgttgtgcggcagttttcagacaatgcagggataacaccagcaatatcattggacctaatgactgatgctgaattggccagagctgtatcatacatgccaacatctgcaggacagataaaactgatgttggagaaccgcgcaatggtaaggagaaaaggatttggaatcctaataggggtctacggaagctctgtgatttacatggttcaattgccgatctttggtgtcatagatacaccttgttggataatcaaggcagctccctcttgctcagaaaaaaacgggaattatgcttgcctcctaagagaggatcaagggtggtattgtaaaaatgcaggatccactgtttactacccaaatgaaaaagactgtgaaacaagaggtgatcatgttttttgtgacacagcagcagggatcaatgttgctgagcaatcaagagaatgcaacatcaacatatctactaccaactacccatgcaaagtcagcacaggaagacaccctataagcatggttgcactatcacctctcggtgctttggtggcttgctataaaggggtaagctgctcgattggcagcaatcgagttggaatcatcaaacaattacctaaaggctgctcatacataactaaccaggatgcagacactgtaacaattgacaataccgtgtatcaactaagcaaagttgaaggtgaacagcatgtaataaaagggagaccagtttcaagcagttttgatccaatcaggtttcctgaggatcagttcaatgttgcgcttgatcaagtcttcgaaagcattgagaacagtcaggcactggtggaacagtcaaacaaaattctaaacagtgcagaaaaaggaaacactggtttcattattgtaataattttggttgctgttcttggtttaaccatgatttcagtgagcatcatcatcataatcaagaaaacaaggaagcccacaggagcacctccagagctgaatggtgtcaccaacggcggtttcataccacatagtagtctagagggcccgcggttcgaaggtaagcctatccctaaccctctcctcggtctcgattctacgtagcggccgc

SEQ ID NO: 134 - protein, 572 aa, Artificial Sequence
  HA-V5-Full
MSWKVMIIISLLITPQHGTMYPYDVPDYALKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIRFPEDQFNVALDQVFESIENSQALVEQSNKILNSAEKGNTGFIIVIILVAVLGLTMISVSIIIIIKKTRKPTGAPPELNGVTNGGFIPHSSLEGPRFEGKPIPNPLLGLDST

(19) United States
(12) Patent Application Publication
(10) Pub. No.: US 2015/0322137 A1
(43) Pub. Date: Nov. 12, 2015

(54) HUMAN ANTIBODY SPECIFIC TO HUMAN METAPNEUMOVIRUS, OR ANTIGEN-BINDING FRAGMENT THEREOF

(71) Applicant: EVEC INC., Hokkaido (JP)

(72) Inventors: Kenzo TAKADA, Hokkaido (JP); Kantou NAKAJIMA, Hokkaido (JP)

(21) Appl. No.: 14/761,879

(22) PCT Filed: Jan. 28, 2014
(86) PCT No.: PCT/JP2014/051866

§ 371 Date: Jul. 17, 2015

(30) Foreign Application Priority Data
Jan. 28, 2013 (JP) 2013-013599

(51) Int. Cl. C07K 16/10

(52) U.S. Cl. CPC C07K 16/1027; C07K 2317/76; C07K 2317/565; C07K 2317/56; C07K 2317/92; C07K 2317/30

(57) ABSTRACT

The present invention provides a novel human-derived monoclonal antibody specifically binding to human metapneumovirus F protein and neutralizing the human metapneumovirus, and an antigen-binding fragment thereof. The present invention also provides a pharmaceutical composition comprising the antibody or an antigen-binding fragment thereof.

### TECHNICAL FIELD

The present invention relates to a monoclonal antibody specifically binding to human metapneumovirus F protein and neutralizing the biological activity of the human metapneumovirus, and an antigen-binding fragment thereof.

### BACKGROUND ART

Respiratory infection is one of the major diseases in humans in terms of morbidity and death rate. Particularly, in children below the age of 5, the respiratory infection is ranked in one of the leading causes of death. In the USA, the respiratory infection, including pneumonia, influenza, and influenza-like diseases, kills 45,000 or more people every year, and a yearly medical cost reportedly reaches 14.6 billion dollars (Non Patent Literature 1).

Respiratory syncytial virus (RSV), parainfluenza virus, influenza virus, coronavirus, rhinovirus, adenovirus, and the like are known as causative viruses of respiratory infection. Recently, viruses causing respiratory infection, including human metapneumovirus (hMPV), SARS coronavirus, and human bocavirus, have been discovered one after another. Among them, hMPV is a virus that was separated and identified for the first time by a Dutch research group in 2001 (Non Patent Literature 2). This hMPV virus had always been found universally in humans, but had been difficult to separate and therefore not been discovered.

The human metapneumovirus (hMPV), a single-stranded (negative strand) RNA virus, is classified into the genus Metapneumovirus within the subfamily Paramyxoviridae of the family Paramyxoviridae and has eight genes respectively encoding N protein, P protein, M protein, F protein, M2 protein, SH protein, G protein, and L protein (Non Patent Literature 2). The gene sequence of hMPV is most analogous to that of avian pneumovirus (APV) and is also analogous to RSV in terms of gene sequence and clinical symptoms among human viruses (Non Patent Literature 3). hMPV is classified according to serological characteristics into two groups, A and B, which are further classified into their two respective subgroups (A1 and A2, and B1 and B2) (Non Patent Literature 4). Among them, the strain A2 is the mainstream in pandemic outbreaks (Non Patent Literature 5). Although the F protein, the G protein, and the SH protein reside on the virus surface, the main neutralizing antibody against hMPV present in serum has been reported to be an antibody against the F protein (Non Patent Literatures 6 and 7).

According to investigation conducted on pediatric patients with respiratory infection below the age of 5 by the group of Williams et al., it has been reported that 20% of the patients with infection of the lower respiratory tract and 15% of the patients with infection of the upper respiratory tract were infected by hMPV (Non Patent Literature 7). Six-month-old to 1-year-old infants having low antibody titers in blood, immune-compromised senior citizens, or immunodeficient patients are at the risk of increase in severity and require particular attention.

Although methods for examining hMPV have already been established, symptomatic treatment is currently the basic treatment of hMPV in the absence of effective antivirus drugs. Research has been conducted on, for example, the development of antibody drugs specific for hMPV F protein (Patent Literatures 1 and 2, and Non Patent Literature 9) and vaccines prepared using attenuated viruses or the like (Patent Literatures 3 and 4) as therapeutic or preventive drugs for hMPV. A human antibody binding both to the F proteins of hMPV and RSV (Patent Literature 5) has been further reported recently. Any of these approaches have only just begun.

### CITATION LIST

### Patent Literature

Patent Literature 1: WO2006/110214
Patent Literature 2: WO2008/043052
Patent Literature 3: WO2002/057302
Patent Literature 4: WO2003/072719
Patent Literature 5: WO2013/140247

### Non Patent Literature

Non Patent Literature 1: Clin. Micro. Reviews, 2006 (vol. 19) p. 546
Non Patent Literature 2: Nature Medicine, 2001 (vol. 7) p. 719
Non Patent Literature 3: Virology, 2002 (vol. 295) p. 119
Non Patent Literature 4: Emerg. Infect. Dis., 2004 (vol. 10) p. 658
Non Patent Literature 5: PLoS ONE, 2012 (vol. 7) e34544
Non Patent Literature 6: J. Virol., 2004 (vol. 78) p. 6927
Non Patent Literature 7: J. Gen. Virol., 2004 (vol. 85) p. 1655
Non Patent Literature 8: New Eng. J. Med., 2004 (vol. 350) p. 443
Non Patent Literature 9: J. Virol., 2006 (vol. 80) p. 7799

### SUMMARY OF INVENTION

### Technical Problem

Human metapneumovirus (hMPV), which was revealed as a causative virus of respiratory infection in 2001, causes this disease in 15 to 20% of pediatric patients with respiratory infection below the age of 5 and incurs the risk of increase in severity, particularly, in 6-month-old to 1-year-old infants, immune-compromised senior citizens, or immunodeficient patients. Incidentally, RSV is found as a human virus analogous to hMPV, and an antibody drug (Synagis) specifically recognizing RSV F protein has been approved as a preventive drug for the disease and is sold in many countries. Still, the treatment of hMPV is merely symptomatic treatment. Meanwhile, for example, research on vaccines prepared using attenuated viruses or the like (Patent Literatures 3 and 4) and research on antibody drugs specific for hMPV F protein have been reported. Only a human Fab antibody DSλ7, etc., recognizing the F protein of the virus, as with Synagis, (Patent Literature 2) has been reported as an example of a human-derived antibody.

The antibody DSλ7, etc., has still low activity in light of affinity, neutralizing activity, or the like, and is still susceptible to improvement as a drug candidate. Particularly, antibodies having high neutralizing activity against a wide range of strains, A1, A2, B1, and B2, if possible, antibodies having high neutralizing activity against the hMPV_A2 strain, which is the mainstream in recent pandemic outbreaks, have been demanded as antibody drugs for hMPV.

In addition, antibodies differing in epitope are also necessary in consideration of the resistance of the virus. All of chimeric antibodies and humanized antibodies have problems associated with immunogenicity. Completely human-derived antibodies have also been strongly demanded as drug candidate anti-hMPV antibodies.

### Solution to Problem

Under these circumstances, the present inventors have conducted diligent studies with the aim of preparing human-derived monoclonal antibodies that exhibit effectiveness equal to or higher than ever at lower doses. As a result, the present inventors have successfully prepared a plurality of antibodies that specifically bind to hMPV F protein, have high neutralizing activity against a wide range of hMPV strains, and are classified into some different groups by epitope grouping. In this way, the present invention has been completed.

Specifically, the present invention relates to a human monoclonal antibody specific for human metapneumovirus (hMPV) F protein or an antigen-binding fragment thereof, a nucleic acid (polynucleotide) encoding the antibody or an antigen-binding fragment thereof, an expression vector containing the nucleic acid, a host cell containing the vector, and a pharmaceutical composition comprising the antibody of the present invention or an antigen-binding fragment, as described below in [1] to [41].

[1] An antibody capable of specifically binding to human metapneumovirus F protein and neutralizing its biological activity, or an antigen-binding fragment thereof, comprising a heavy chain variable region and a light chain variable region, wherein
(i) the heavy chain variable region comprises:
(a) the amino acid sequence of heavy chain CDR1 selected from the group consisting of the amino acid sequences of SEQ ID NOs: 3, 13, 23, 33, 43, 53, 63, 73, 83, 93, 103, 113, and 123 and amino acid sequences derived from any of these amino acid sequences by the deletion, substitution, insertion, and/or addition mutations of one to several amino acid residues,
(b) the amino acid sequence of heavy chain CDR2 selected from the group consisting of the amino acid sequences of SEQ ID NOs: 4, 14, 24, 34, 44, 54, 64, 74, 84, 94, 104, 114, and 124 and amino acid sequences derived from any of these amino acid sequences by the deletion, substitution, insertion, and/or addition mutations of one to several amino acid residues, and
(c) the amino acid sequence of heavy chain CDR3 selected from the group consisting of the amino acid sequences of SEQ ID NOs: 5, 15, 25, 35, 45, 55, 65, 75, 85, 95, 105, 115, and 125 and amino acid sequences derived from any of these amino acid sequences by the deletion, substitution, insertion, and/or addition mutations of one to several amino acid residues, and
(ii) the light chain variable region comprises:
(a) the amino acid sequence of light chain CDR1 selected from the group consisting of the amino acid sequences of SEQ ID NOs: 8, 18, 28, 38, 48, 58, 68, 78, 88, 98, 108, 118, and 128 and amino acid sequences derived from any of these amino acid sequences by the deletion, substitution, insertion, and/or addition mutations of one to several amino acid residues,
(b) the amino acid sequence of light chain CDR2 selected from the group consisting of the amino acid sequences of SEQ ID NOs: 9, 19, 29, 39, 49, 59, 69, 79, 89, 99, 109, 119, and 129 and amino acid sequences derived from any of these amino acid sequences by the deletion, substitution, insertion, and/or addition mutations of one to several amino acid residues, and
(c) the amino acid sequence of light chain CDR3 selected from the group consisting of the amino acid sequences of SEQ ID NOs: 10, 20, 30, 40, 50, 60, 70, 80, 90, 100, 110, 120, and 130 and amino acid sequences derived from any of these amino acid sequences by the deletion, substitution, insertion, and/or addition mutations of one to several amino acid residues.
[2] The antibody or an antigen-binding fragment thereof according to [1], wherein
(i) the heavy chain variable region comprises:
(a) the amino acid sequence of heavy chain CDR1 comprising the amino acid sequence of SEQ ID NO: 3,
(b) the amino acid sequence of heavy chain CDR2 comprising the amino acid sequence of SEQ ID NO: 4, and
(c) the amino acid sequence of heavy chain CDR3 comprising the amino acid sequence of SEQ ID NO: 5, and
(ii) the light chain variable region comprises:
(a) the amino acid sequence of light chain CDR1 comprising the amino acid sequence of SEQ ID NO: 8,
(b) the amino acid sequence of light chain CDR2 comprising the amino acid sequence of SEQ ID NO: 9, and
(c) the amino acid sequence of light chain CDR3 comprising the amino acid sequence of SEQ ID NO: 10.
[3] The antibody or an antigen-binding fragment thereof according to [1], wherein
(i) the heavy chain variable region comprises
(a) the amino acid sequence of heavy chain CDR1 comprising the amino acid sequence of SEQ ID NO: 13,
(b) the amino acid sequence of heavy chain CDR2 comprising the amino acid sequence of SEQ ID NO: 14, and
(c) the amino acid sequence of heavy chain CDR3 comprising the amino acid sequence of SEQ ID NO: 15, and
(ii) the light chain variable region comprises
(a) the amino acid sequence of light chain CDR1 comprising the amino acid sequence of SEQ ID NO: 18,
(b) the amino acid sequence of light chain CDR2 comprising the amino acid sequence of SEQ ID NO: 19, and
(c) the amino acid sequence of light chain CDR3 comprising the amino acid sequence of SEQ ID NO: 20.
[4] The antibody or an antigen-binding fragment thereof according to [1], wherein
(i) the heavy chain variable region comprises
(a) the amino acid sequence of heavy chain CDR1 comprising the amino acid sequence of SEQ ID NO: 23,
(b) the amino acid sequence of heavy chain CDR2 comprising the amino acid sequence of SEQ ID NO: 24, and
(c) the amino acid sequence of heavy chain CDR3 comprising the amino acid sequence of SEQ ID NO: 25, and
(ii) the light chain variable region comprises
(a) the amino acid sequence of light chain CDR1 comprising the amino acid sequence of SEQ ID NO: 28,
(b) the amino acid sequence of light chain CDR2 comprising the amino acid sequence of SEQ ID NO: 29, and
(c) the amino acid sequence of light chain CDR3 comprising the amino acid sequence of SEQ ID NO: 30.
[5] The antibody or an antigen-binding fragment thereof according to [1], wherein
(i) the heavy chain variable region comprises
(a) the amino acid sequence of heavy chain CDR1 comprising the amino acid sequence of SEQ ID NO: 33,
(b) the amino acid sequence of heavy chain CDR2 comprising the amino acid sequence of SEQ ID NO: 34, and
(c) the amino acid sequence of heavy chain CDR3 comprising the amino acid sequence of SEQ ID NO: 35, and
(ii) the light chain variable region comprises
(a) the amino acid sequence of light chain CDR1 comprising the amino acid sequence of SEQ ID NO: 38,
(b) the amino acid sequence of light chain CDR2 comprising the amino acid sequence of SEQ ID NO: 39, and
(c) the amino acid sequence of light chain CDR3 comprising the amino acid sequence of SEQ ID NO: 40.
[6] The antibody or an antigen-binding fragment thereof according to [1], wherein
(i) the heavy chain variable region comprises
(a) the amino acid sequence of heavy chain CDR1 comprising the amino acid sequence of SEQ ID NO: 43,
(b) the amino acid sequence of heavy chain CDR2 comprising the amino acid sequence of SEQ ID NO: 44, and
(c) the amino acid sequence of heavy chain CDR3 comprising the amino acid sequence of SEQ ID NO: 45, and
(ii) the light chain variable region comprises
(a) the amino acid sequence of light chain CDR1 comprising the amino acid sequence of SEQ ID NO: 48,
(b) the amino acid sequence of light chain CDR2 comprising the amino acid sequence of SEQ ID NO: 49, and
(c) the amino acid sequence of light chain CDR3 comprising the amino acid sequence of SEQ ID NO: 50.
[7] The antibody or an antigen-binding fragment thereof according to [1], wherein
(i) the heavy chain variable region comprises
(a) the amino acid sequence of heavy chain CDR1 comprising the amino acid sequence of SEQ ID NO: 53,
(b) the amino acid sequence of heavy chain CDR2 comprising the amino acid sequence of SEQ ID NO: 54, and
(c) the amino acid sequence of heavy chain CDR3 comprising the amino acid sequence of SEQ ID NO: 55, and
(ii) the light chain variable region comprises
(a) the amino acid sequence of light chain CDR1 comprising the amino acid sequence of SEQ ID NO: 58,
(b) the amino acid sequence of light chain CDR2 comprising the amino acid sequence of SEQ ID NO: 59, and
(c) the amino acid sequence of light chain CDR3 comprising the amino acid sequence of SEQ ID NO: 60.
[8] The antibody or an antigen-binding fragment thereof according to [1], wherein
(i) the heavy chain variable region comprises
(a) the amino acid sequence of heavy chain CDR1 comprising the amino acid sequence of SEQ ID NO: 63,
(b) the amino acid sequence of heavy chain CDR2 comprising the amino acid sequence of SEQ ID NO: 64, and
(c) the amino acid sequence of heavy chain CDR3 comprising the amino acid sequence of SEQ ID NO: 65, and
(ii) the light chain variable region comprises
(a) the amino acid sequence of light chain CDR1 comprising the amino acid sequence of SEQ ID NO: 68,
(b) the amino acid sequence of light chain CDR2 comprising the amino acid sequence of SEQ ID NO: 69, and
(c) the amino acid sequence of light chain CDR3 comprising the amino acid sequence of SEQ ID NO: 70.
[9] The antibody or an antigen-binding fragment thereof according to [1], wherein
(i) the heavy chain variable region comprises
(a) the amino acid sequence of heavy chain CDR1 comprising the amino acid sequence of SEQ ID NO: 73,
(b) the amino acid sequence of heavy chain CDR2 comprising the amino acid sequence of SEQ ID NO: 74, and
(c) the amino acid sequence of heavy chain CDR3 comprising the amino acid sequence of SEQ ID NO: 75, and
(ii) the light chain variable region comprises
(a) the amino acid sequence of light chain CDR1 comprising the amino acid sequence of SEQ ID NO: 78,
(b) the amino acid sequence of light chain CDR2 comprising the amino acid sequence of SEQ ID NO: 79, and
(c) the amino acid sequence of light chain CDR3 comprising the amino acid sequence of SEQ ID NO: 80.
[10] The antibody or an antigen-binding fragment thereof according to [1], wherein
(i) the heavy chain variable region comprises
(a) the amino acid sequence of heavy chain CDR1 comprising the amino acid sequence of SEQ ID NO: 83,
(b) the amino acid sequence of heavy chain CDR2 comprising the amino acid sequence of SEQ ID NO: 84, and
(c) the amino acid sequence of heavy chain CDR3 comprising the amino acid sequence of SEQ ID NO: 85, and
(ii) the light chain variable region comprises
(a) the amino acid sequence of light chain CDR1 comprising the amino acid sequence of SEQ ID NO: 88,
(b) the amino acid sequence of light chain CDR2 comprising the amino acid sequence of SEQ ID NO: 89, and
(c) the amino acid sequence of light chain CDR3 comprising the amino acid sequence of SEQ ID NO: 90.
[11] The antibody or an antigen-binding fragment thereof according to [1], wherein
(i) the heavy chain variable region comprises
(a) the amino acid sequence of heavy chain CDR1 comprising the amino acid sequence of SEQ ID NO: 93,
(b) the amino acid sequence of heavy chain CDR2 comprising the amino acid sequence of SEQ ID NO: 94, and
(c) the amino acid sequence of heavy chain CDR3 comprising the amino acid sequence of SEQ ID NO: 95, and
(ii) the light chain variable region comprises
(a) the amino acid sequence of light chain CDR1 comprising the amino acid sequence of SEQ ID NO: 98,
(b) the amino acid sequence of light chain CDR2 comprising the amino acid sequence of SEQ ID NO: 99, and
(c) the amino acid sequence of light chain CDR3 comprising the amino acid sequence of SEQ ID NO: 100.
[12] The antibody or an antigen-binding fragment thereof according to [1], wherein
(i) the heavy chain variable region comprises
(a) the amino acid sequence of heavy chain CDR1 comprising the amino acid sequence of SEQ ID NO: 103,
(b) the amino acid sequence of heavy chain CDR2 comprising the amino acid sequence of SEQ ID NO: 104, and
(c) the amino acid sequence of heavy chain CDR3 comprising the amino acid sequence of SEQ ID NO: 105, and
(ii) the light chain variable region comprises
(a) the amino acid sequence of light chain CDR1 comprising the amino acid sequence of SEQ ID NO: 108,
(b) the amino acid sequence of light chain CDR2 comprising the amino acid sequence of SEQ ID NO: 109, and
(c) the amino acid sequence of light chain CDR3 comprising the amino acid sequence of SEQ ID NO: 110.
[13] The antibody or an antigen-binding fragment thereof according to [1], wherein
(i) the heavy chain variable region comprises
(a) the amino acid sequence of heavy chain CDR1 comprising the amino acid sequence of SEQ ID NO: 113,
(b) the amino acid sequence of heavy chain CDR2 comprising the amino acid sequence of SEQ ID NO: 114, and
(c) the amino acid sequence of heavy chain CDR3 comprising the amino acid sequence of SEQ ID NO: 115, and
(ii) the light chain variable region comprises
(a) the amino acid sequence of light chain CDR1 comprising the amino acid sequence of SEQ ID NO: 118,
(b) the amino acid sequence of light chain CDR2 comprising the amino acid sequence of SEQ ID NO: 119, and
(c) the amino acid sequence of light chain CDR3 comprising the amino acid sequence of SEQ ID NO: 120.
[14] The antibody or an antigen-binding fragment thereof according to [1], wherein
(i) the heavy chain variable region comprises
(a) the amino acid sequence of heavy chain CDR1 comprising the amino acid sequence of SEQ ID NO: 123,
(b) the amino acid sequence of heavy chain CDR2 comprising the amino acid sequence of SEQ ID NO: 124, and
(c) the amino acid sequence of heavy chain CDR3 comprising the amino acid sequence of SEQ ID NO: 125, and
(ii) the light chain variable region comprises
(a) the amino acid sequence of light chain CDR1 comprising the amino acid sequence of SEQ ID NO: 128,
(b) the amino acid sequence of light chain CDR2 comprising the amino acid sequence of SEQ ID NO: 129, and
(c) the amino acid sequence of light chain CDR3 comprising the amino acid sequence of SEQ ID NO: 130.
[15] The antibody or an antigen-binding fragment thereof according to any one of [1] to [14], wherein the antibody comprises
(a) a heavy chain variable region (VH) selected from the group consisting of the amino acid sequences of SEQ ID NOs: 2, 12, 22, 32, 42, 52, 62, 72, 82, 92, 102, 112, and 122 and amino acid sequences having 95% or higher identity to any of these amino acid sequences, and
(b) a light chain variable region (VL) selected from the group consisting of the amino acid sequences of SEQ ID NOs: 7, 17, 27, 37, 47, 57, 67, 77, 87, 97, 107, 117, and 127 and amino acid sequences having 95% or higher identity to any of these amino acid sequences.
[16] The antibody or an antigen-binding fragment thereof according to [15], wherein the antibody comprises
(a) a heavy chain variable region (VH) comprising the amino acid sequence of SEQ ID NO: 2 or an amino acid sequence having 95% or higher identity to the amino acid sequence of SEQ ID NO: 2, and
(b) a light chain variable region (VL) comprising the amino acid sequence of SEQ ID NO: 7 or an amino acid sequence having 95% or higher identity to the amino acid sequence of SEQ ID NO: 7.
[17] The antibody or an antigen-binding fragment thereof according to [15], wherein the antibody comprises
(a) a heavy chain variable region (VH) comprising the amino acid sequence of SEQ ID NO: 12 or an amino acid sequence having 95% or higher identity to the amino acid sequence of SEQ ID NO: 12, and
(b) a light chain variable region (VL) comprising the amino acid sequence of SEQ ID NO: 17 or an amino acid sequence having 95% or higher identity to the amino acid sequence of SEQ ID NO: 17.
[18] The antibody or an antigen-binding fragment thereof according to [15], wherein the antibody comprises
(a) a heavy chain variable region (VH) comprising the amino acid sequence of SEQ ID NO: 22 or an amino acid sequence having 95% or higher identity to the amino acid sequence of SEQ ID NO: 22, and
(b) a light chain variable region (VL) comprising the amino acid sequence of SEQ ID NO: 27 or an amino acid sequence having 95% or higher identity to the amino acid sequence of SEQ ID NO: 27.
[19] The antibody or an antigen-binding fragment thereof according to [15], wherein the antibody comprises
(a) a heavy chain variable region (VH) comprising the amino acid sequence of SEQ ID NO: 32 or an amino acid sequence having 95% or higher identity to the amino acid sequence of SEQ ID NO: 32, and
(b) a light chain variable region (VL) comprising the amino acid sequence of SEQ ID NO: 37 or an amino acid sequence having 95% or higher identity to the amino acid sequence of SEQ ID NO: 37.
[20] The antibody or an antigen-binding fragment thereof according to [15], wherein the antibody comprises
(a) a heavy chain variable region (VH) comprising the amino acid sequence of SEQ ID NO: 42 or an amino acid sequence having 95% or higher identity to the amino acid sequence of SEQ ID NO: 42, and
(b) a light chain variable region (VL) comprising the amino acid sequence of SEQ ID NO: 47 or an amino acid sequence having 95% or higher identity to the amino acid sequence of SEQ ID NO: 47.
[21] The antibody or an antigen-binding fragment thereof according to [15], wherein the antibody comprises
(a) a heavy chain variable region (VH) comprising the amino acid sequence of SEQ ID NO: 52 or an amino acid sequence having 95% or higher identity to the amino acid sequence of SEQ ID NO: 52, and
(b) a light chain variable region (VL) comprising the amino acid sequence of SEQ ID NO: 57 or an amino acid sequence having 95% or higher identity to the amino acid sequence of SEQ ID NO: 57.
[22] The antibody or an antigen-binding fragment thereof according to [15], wherein the antibody comprises
(a) a heavy chain variable region (VH) comprising the amino acid sequence of SEQ ID NO: 62 or an amino acid sequence having 95% or higher identity to the amino acid sequence of SEQ ID NO: 62, and
(b) a light chain variable region (VL) comprising the amino acid sequence of SEQ ID NO: 67 or an amino acid sequence having 95% or higher identity to the amino acid sequence of SEQ ID NO: 67.
[23] The antibody or an antigen-binding fragment thereof according to [15], wherein the antibody comprises
(a) a heavy chain variable region (VH) comprising the amino acid sequence of SEQ ID NO: 72 or an amino acid sequence having 95% or higher identity to the amino acid sequence of SEQ ID NO: 72, and
(b) a light chain variable region (VL) comprising the amino acid sequence of SEQ ID NO: 77 or an amino acid sequence having 95% or higher identity to the amino acid sequence of SEQ ID NO: 77.
[24] The antibody or an antigen-binding fragment thereof according to [15], wherein the antibody comprises
(a) a heavy chain variable region (VH) comprising the amino acid sequence of SEQ ID NO: 82 or an amino acid sequence having 95% or higher identity to the amino acid sequence of SEQ ID NO: 82, and
(b) a light chain variable region (VL) comprising the amino acid sequence of SEQ ID NO: 87 or an amino acid sequence having 95% or higher identity to the amino acid sequence of SEQ ID NO: 87.
[25] The antibody or an antigen-binding fragment thereof according to [15], wherein the antibody comprises
(a) a heavy chain variable region (VH) comprising the amino acid sequence of SEQ ID NO: 92 or an amino acid sequence having 95% or higher identity to the amino acid sequence of SEQ ID NO: 92, and
(b) a light chain variable region (VL) comprising the amino acid sequence of SEQ ID NO: 97 or an amino acid sequence having 95% or higher identity to the amino acid sequence of SEQ ID NO: 97.
[26] The antibody or an antigen-binding fragment thereof according to [15], wherein the antibody comprises
(a) a heavy chain variable region (VH) comprising the amino acid sequence of SEQ ID NO: 102 or an amino acid sequence having 95% or higher identity to the amino acid sequence of SEQ ID NO: 102, and
(b) a light chain variable region (VL) comprising the amino acid sequence of SEQ ID NO: 107 or an amino acid sequence having 95% or higher identity to the amino acid sequence of SEQ ID NO: 107.
[27] The antibody or an antigen-binding fragment thereof according to [15], wherein the antibody comprises
(a) a heavy chain variable region (VH) comprising the amino acid sequence of SEQ ID NO: 112 or an amino acid sequence having 95% or higher identity to the amino acid sequence of SEQ ID NO: 112, and
(b) a light chain variable region (VL) comprising the amino acid sequence of SEQ ID NO: 117 or an amino acid sequence having 95% or higher identity to the amino acid sequence of SEQ ID NO: 117.
[28] The antibody or an antigen-binding fragment thereof according to [15], wherein the antibody comprises
(a) a heavy chain variable region (VH) comprising the amino acid sequence of SEQ ID NO: 122 or an amino acid sequence having 95% or higher identity to the amino acid sequence of SEQ ID NO: 122, and
(b) a light chain variable region (VL) comprising the amino acid sequence of SEQ ID NO: 127 or an amino acid sequence having 95% or higher identity to the amino acid sequence of SEQ ID NO: 127.
[29] The antibody or an antigen-binding fragment thereof according to any one of [1] to [28], wherein the epitope of the antibody belongs to any of epitope groups Group 1 typified by anti-hMPV antibodies EV046115b, EV046130, and EV046147, Group 2 typified by anti-hMPV antibodies EV046113, EV046116, EV046141, and EV0461142, Group 3 typified by anti-hMPV antibodies EV046124, EV046143, and EV046150, Group 4 typified by anti-hMPV antibodies EV046120 and EV046135, and Group 5 typified by an anti-hMPV antibody EV046136 in epitope grouping according to binding activity against a partially deleted peptide of the F protein.
[30] The antibody or an antigen-binding fragment thereof according to any one of [1] to [28], wherein the epitope of the antibody belongs to any of epitope groups Group 2 typified by anti-hMPV antibodies EV046113, EV046116, EV046141, and EV0461142, Group 3 typified by anti-hMPV antibodies EV046124, EV046143, and EV046150, Group 4 typified by anti-hMPV antibodies EV046120 and EV046135, and Group 5 typified by an anti-hMPV antibody EV046136 in epitope grouping according to binding activity against a partially deleted peptide of the F protein.
[31] The antibody or an antigen-binding fragment thereof according to any one of [1] to [28], wherein the epitope of the antibody belongs to an epitope group Group 3 typified by anti-hMPV antibodies EV046124, EV046143, and EV046150 in epitope grouping according to binding activity against a partially deleted peptide of the F protein.
[32] The antibody or an antigen-binding fragment thereof according to any one of [1] to [31], wherein the antibody is of IgG1 (K) or IgG1 (λ) class (subclass).
[33] The antibody or an antigen-binding fragment thereof according to any one of [1] to [32], wherein the antibody has a neutralizing activity (IC50) of approximately 1 μg/mL (approximately 6.7 nM) or lower against all of human metapneumovirus strains JPS02-76 (type B1), JPS05-21 (type B2), and JPS03-180 (type A1).
[34] The antibody or an antigen-binding fragment thereof according to any one of [1] to [32], wherein the antibody has a neutralizing activity (IC50) of approximately 1 μg/mL (approximately 6.7 nM) or lower against all of human metapneumovirus strains JPS03-180 (type A1), JPS03-178 (type A2), JPS02-76 (type B1), and JPS05-21 (type B2).
[35] The antibody or an antigen-binding fragment thereof according to any one of [1] to [32], wherein the antibody has a neutralizing activity (IC90) of approximately 2 μg/mL (approximately 13.3 nM) or lower against all of human metapneumovirus strains JPS03-180 (type A1), JPS03-178 (type A2), JPS02-76 (type B1), and JPS05-21 (type B2).
[36] The antibody or an antigen-binding fragment thereof according to any one of [1] to [32], wherein the antibody has a neutralizing activity (IC50) of approximately 0.1 μg/mL (approximately 0.67 nM) or lower against a human metapneumovirus JPS03-178 strain (type A2).
[37] A pharmaceutical composition comprising an antibody or an antigen-binding fragment thereof according to any one of [1] to [36] and a pharmaceutically acceptable carrier.
[38] The pharmaceutical composition according to [37], wherein the pharmaceutical composition is intended for the treatment or prevention of human metapneumovirus infection.
[39] An isolated nucleic acid encoding the amino acid sequence of an antibody or an antigen-binding fragment thereof according to any one of [1] to [36], an isolated nucleic acid encoding an amino acid sequence of any of SEQ ID NOs: 1, 6, 11, 16, 21, 26, 31, 36, 41, 46, 51, 56, 61, 66, 71, 76, 81, 86, 91, 96, 101, 106, 111, 116, 121, and 126, or an isolated nucleic acid hybridizing under high stringent conditions to any of these nucleic acids.
[40] A recombinant expression vector comprising an isolated nucleic acid according to [39] incorporated therein.
[41] A host cell comprising a recombinant expression vector according to [40] introduced thereinto.

### Advantageous Effects of Invention

The monoclonal antibody neutralizing the biological activity of human metapneumovirus (hMPV) according to the present invention or an antigen-binding fragment thereof specifically binds to human metapneumovirus (hMPV) F protein and cancels (neutralizes) its biological activity. Thus, the monoclonal antibody according to the present invention or an antigen-binding fragment thereof can be expected to prevent or treat hMPV infection. In an embodiment, the monoclonal antibody according to the present invention or an antigen-binding fragment thereof is a human monoclonal antibody against human metapneumovirus (hMPV) F protein or an antigen-binding fragment thereof and therefore, is advantageously neither immunogenic nor causes immune response. A pharmaceutical composition comprising the particularly preferred human monoclonal antibody according to the present invention is effective in a very small amount and can also be expected to reduce medical cost.

### BRIEF DESCRIPTION OF DRAWINGS

FIG. 1 is a diagram showing, in the form of flow chart, procedures of separating an antibody-producing cell clone producing the anti-hMPV antibody according to the present invention.

FIG. 2 is a diagram showing the results of conducting the competition assay of anti-MPV antibodies using Biacore T-200. FP-TM(−)-His, which was His-Tag-added hMPV (JPS02-76) FP lacking the TM region (a.a. 491-539), was captured onto Sensor Chip NTA. An anti-MPV antibody was bound as Sample 1 to the sensor chip until a saturation level. Next, another anti-MPV antibody was bound thereto as Sample 2. The presence or absence of competition was determined on the basis of whether or not the binding of the anti-hMPV antibody as Sample 2 was inhibited. Ab. I.D. was indicated by the last 3 digits. The mark + represents competing, and the mark − represents not competing. N.D. represents being immeasurable due to the aggregation reaction of antibodies on the sensor chip. In the diagram, Ab. I.D. 338 and 210 represent mAb388 and HMB3210, respectively, used as controls.

FIG. 3 is a diagram showing various deletion mutants (hMPV-FP deletion mutants) prepared for the epitope mapping of anti-hMPV antibodies. HA-V5-Full is hMPV (JPS02-76) FP containing HA-Tag (HA) added downstream of the signal peptide (SP) and V5-Tag (V5) added downstream of the C-tail (C). Each deletion mutant was deficient in the region shown in the diagram and cloned into an expression vector. Other abbreviations in the diagram are as follows: F2 domain (F2), fusion peptide (FP), heptad repeat 1 (HR1), F1 domain (F1), heptad repeat 2 (HR2), and transmembrane domain (TM).

FIG. 4 is a diagram the showing results of the binding assay of anti-hMPV antibodies to deletion mutants. CHO-K1 cells were transfected with each deletion mutant expression vector shown in FIG. 3, inoculated to a 96-well plate 16 hours later, and fixed 24 hours later. The resulting deletion mutant-expressing CHO-K1 cells were fluorescently immunostained to confirm the binding of each anti-MPV antibody. The details of this assay are similar to those of cell fluorescent immunostaining screening. Also, HA and V5 antibodies were subjected to cell fluorescent immunostaining to confirm the expression of the deletion mutants themselves. Ab. I.D. was indicated by the last 3 digits. The marks +++, ++, and + represent binding in the order of fluorescence intensity. The mark − represents not binding. Epitope group was determined on the basis of binding patterns to the deletion mutants. In the diagram, Ab. I.D. 338 and 210 represent mAb388 and HMB3210, respectively, used as controls.

FIG. 5 is a diagram showing the epitope grouping of anti-hMPV antibodies. The anti-hMPV antibodies were epitope-grouped on the basis of the results shown in FIGS. 3 and 4. The circled groups are based on the competition assay results, and the boxed groups are based on the deletion mutant binding assay results.

### DESCRIPTION OF EMBODIMENTS

### 1. Description of Terms

In the present specification, scientific terms and technical terms used in relation to the present invention have meanings generally understood by those skilled in the art. Words in the singular form shall be construed to include the plural and vice versa, unless the context otherwise requires. In general, nomenclatures used in relation to cells and techniques of tissue culture, molecular biology, immunology, microbiology, genetics, protein and nucleic acid chemistries, and hybridization described herein are well known in the art and generally used.

The present invention relates to a monoclonal antibody specifically binding to human metapneumovirus F protein and neutralizing the biological activity of the human metapneumovirus, and an antigen-binding fragment thereof. Hereinafter, the embodiments of the present invention will be described in detail by defining the terms used in the present invention.

1) Human Metapneumovirus (Also Abbreviated to hMPV)

The human metapneumovirus is one of the causative viruses of human respiratory infection that was discovered by a Dutch research group in 2001. This virus is classified into the genus Metapneumovirus within the subfamily Paramyxoviridae of the family Paramyxoviridae and, currently, further classified into four subtypes (A1, A2, B1, and B2; also referred to as type A1, type A2, type B1, and type B2, respectively) on the basis of results of phylogenetic analysis using the nucleotide sequences of G and F proteins (Non Patent Literature 4). The subgroup A2 was further classified into two types, hMPV_A2a and hMPV_A2b, in Germany in 2006 (Non Patent Literature 5). Analogous viruses are avian pneumovirus (also abbreviated to APV), which infects bird, human respiratory syncytial virus (also abbreviated to RSV), and the like. hMPV, a 13.35-kb single-stranded (negative strand) RNA virus, has eight genes, N, P, M, F, M2, SH, G, and L, and express three glycoproteins, F, G, and SH on the virus surface ((Non Patent Literature 1). Reportedly, an antibody against the F protein accounts for a major proportion of neutralizing antibodies against hMPV present in serum.

2) hMPV F Protein

The F protein (hereinafter, also abbreviated to FP) is one of the glycoproteins present on the virus surface of hMPV. The F proteins in typical strains of four hMPV subgroups (A1, A2, B1, and B2) are each composed of 539 amino acid residues. Their typical amino acid sequences are disclosed in GenBank Accession Nos. AF371337 for A1 (NL/1/00 strain), AY304360 for A2 (NL/17/00 strain), AY304361 for B1 (NL/1/99 strain), and AY304362 for B2 (NL/1/94 strain). The amino acid sequence of the A1 F protein, as compared with other viruses, has 81% identity to the amino acid sequence of the F protein of avian pneumovirus type C and 33% identity to the amino acid sequence of the F protein of human RSV (Non Patent Literature 3), suggesting that the hMPV F protein plays the same role as that of the F proteins of these viruses. Specifically, the hMPV F protein is considered to be a fusion protein that is involved in the fusion between the envelope of the virus attached to a cell and the membrane of the host cell. Thus, it has been reported that antibodies having high neutralizing activity are found among antibodies binding to the hMPV F protein (Patent Literatures 1 and 2). Among antibodies against human RSV F protein, an antibody (Synagis) has already been used as a drug.

When the amino acid sequence of the F protein is compared among the four hMPV subgroups, the amino acid sequence of each subgroup (A1, A2, B1, and B2) has 99 to 100% identity to each other and has 97 to 99% identity between the subtypes (between A1 and A2 or between B1 and B2). When the amino acid sequence is compared between groups A and B, 94 to 97% identity is confirmed. Thus, the amino acid sequence of the hMPV F protein is known to be highly conserved among the groups (Non Patent Literature 4). In the present invention, the amino acid sequence of the F protein of a JPS02-76 strain (B1; T285I) used in the screening evaluation of human antibodies exhibited identity as very high as 94% to the amino acid sequence of the A1 (NL/1/00 strain) F protein, 94% to the amino acid sequence of the A2 (NL/17/00 strain) F protein, 99% to the B1 (NL/1/99 strain) F protein, and 99% to the B2 (NL/1/94 strain) F protein, as compared with the typical F proteins mentioned above. This strongly suggests that the evaluation system using the F protein of the JPS02-76 strain used in the present invention is a method capable of obtaining antibodies cross-reactive with the hMPV strain of any subgroup.

3) Antibody Binding to hMPV F Protein

This antibody specifically binds to human metapneumovirus (hMPV) FP and is capable of binding to an epitope site, for example, a linear epitope, a discontinuous epitope, or a conformational epitope, in the FP, or a fragment of the FP, etc. In the present specification, the “anti-hMPV antibody”, the “antibody capable of neutralizing hMPV”, the “anti-hMPV-F protein antibody”, the “antibody specifically binding to human metapneumovirus (hMPV) FP”, or the “antibody capable of neutralizing the biological activity of human metapneumovirus (hMPV)” refers to an antibody that inhibits the biological activity of the human metapneumovirus (hMPV) through binding to the hMPV FP. As mentioned above, since the identity of FP is very high among the hMPV subtypes, the antibody specifically binding to hMPV FP is generally screened for by a method using FP of any of the typical strains of four hMPV subgroups (A1, A2, B1, and B2) as an antigen. In the present invention, the FP gene was cloned from the hMPV B1 strain (JPS02-76) and expressed in CHO-K1 cells, and the resulting FP was used. The nucleotide sequence of the obtained FP gene (GenBank Accession No. AY530089) and the amino acid sequence of FP (GenBank Accession No. AAS22077) are shown in SEQ ID NOs: 131 and 132, respectively.

### 4) Antibody

The term “antibody” used herein refers to an immunoglobulin molecule composed of four polypeptide chains, i.e., two heavy (H) chains and two light (L) chains, linked via disulfide bonds. The monoclonal antibody according to the present invention is also composed of an immunoglobulin molecule comprising two heavy chains (H chains) and two light chains (L chains). Each H chain is composed of an H chain variable part region (also referred to as “HCVR” or “VH”) and an H chain invariable part region (the H chain invariable part region is composed of three domains, which are also referred to as “CH1”, “CH2”, and “CH3”, respectively (collectively referred to as CH)). Each L chain is composed of an L chain variable part region (also referred to as “LCVR” or “VL”) and an L chain invariable part region (the L chain invariable part region is composed of one domain, which is also referred to as “CL”). A region located before each invariable part region (also referred to as invariable region or constant region) is called a variable part region (also referred to as variable region).

Particularly, VH and VL are important because of their involvement in the binding specificity of the antibody. Since the antibody interacts with its target antigen mainly through the amino acid residues of VH and VL, the amino acid sequences within the variable part regions differ more largely among individual antibodies than sequences without the variable part regions. VH and VL can be further subdivided into regions called framework regions (FRs) conserved among various antibodies, and hypervariable regions called complementarity-determining regions (CDRs). Each of VH and VL has three CDRs and four FRs, and these regions are arranged from the amino terminus to the carboxy terminus in the order of FR1, CDR1, FR2, CDR2, FR3, CDR3, and FR4.

The antibody according to the present invention means a whole antibody or an antigen-binding fragment thereof. On the basis of the amino acid sequence representing each variable part region or the amino acid sequence of each complementarity-determining region (CDR), human monoclonal antibodies and monoclonal antibodies (also including chimeric antibodies and humanized antibodies) each capable of specifically binding to hMPV F protein and neutralizing the biological activity of the hMPV, or antigen-binding fragments thereof can be obtained by a technique well known in the art. These antibodies are included in the technical scope of the present invention.

### 5) “Antigen-Binding Fragment” of the Antibody (or Simply Referred to as “Antibody Fragment”)

The term “antigen-binding fragment” of the antibody (or simply referred to as an “antibody fragment”) used herein refers to one or more antibody fragments (e.g., VH) having the ability to specifically bind to the antigen (F proteins of hMPV). The fragment also includes a peptide having the minimum amino acid sequence specifically binding to the antigen. Examples of binding moieties included in the term “antigen-binding fragment” of the antibody include (i) a Fab fragment, (ii) a F(ab′)2 fragment, (iii) an Fd fragment composed of VH and CH1 domains, (iv) an Fv fragment composed of VL and VH domains on a single arm of the antibody, (v) a dAb fragment composed of a VH domain (Ward et al., Nature 341: 544-546, 1989), (vi) isolated complementarity-determining regions (CDRs) having frameworks sufficient for the specific binding, (vii) a bispecific antibody, and (viii) a multispecific antibody. In the present specification, the simple term “antibody” used without discrimination includes not only whole antibodies but also such “antigen-binding fragments”.

### 6) Isotype and Subclass

Heavy chains are divided according to difference in constant region into γ, μ, α, δ, and ε chains. Depending on this difference, 5 types of classes (isotypes), IgG, IgM, IgA, IgD, and IgE, respectively, are formed in immunoglobulins. In the case of human IgG, four subclasses, IgG1 to IgG4, are present. On the other hand, light chains are divided according to difference in constant region into κ and λ chains. The antibody according to the present invention is particularly preferably of IgG1 (κ) or IgG1 (λ) class (subclass).

### 2. Antibody According to the Present Invention or Antigen-Binding Fragment Thereof

In one embodiment, the present invention provides an antibody capable of specifically binding to hMPV and neutralizing the biological activity of the hMPV, or an antigen-binding fragment thereof (hereinafter, referred to as the antibody of the present invention). Typically, the antibody of the present invention is an antibody capable of specifically binding to hMPV F protein and neutralizing the biological activity of the protein, or an antigen-binding fragment thereof.

The anti-hMPV antibody of the present invention includes not only an antibody comprising the amino acid sequence of a particular heavy chain or light chain, its variable region, or its CDRs, etc., shown in SEQ ID NO described in the present specification, but an anti-hMPV antibody comprising an amino acid sequence substantially identical to any of these amino acid sequences and capable of specifically binding to hMPV F protein and neutralizing the biological activity of the hMPV. In this context, the term “substantially identical” means the presence of the deletion, substitution, insertion, or addition of one or more amino acid residues in the amino acid sequence of the protein of the present invention, or the combination of any two or more of these means that one or more amino acid residues are deleted, substituted, inserted, or added at one or more arbitrary positions in the same amino acid sequence, and two or more of the deletion, the substitution, the insertion, and the addition may occur at the same time.

Amino acids constituting proteins in the natural world can be grouped according to the properties of their side chains and can be classified into amino acid groups having similar properties, for example, groups of aromatic amino acids (tyrosine, phenylalanine, and tryptophan), basic amino acids (lysine, arginine, and histidine), acidic amino acids (aspartic acid and glutamic acid), neutral amino acids (serine, threonine, asparagine, and glutamine), hydrocarbon chain-containing amino acids (alanine, valine, leucine, isoleucine, and proline), and others (glycine, methionine, and cysteine).

As an example, amino acid residues that can be mutually substituted, also including nonnatural amino acids, may be grouped as follows, and the amino acid residues included in the same group can be mutually substituted: group A: leucine, isoleucine, norleucine, valine, norvaline, alanine, 2-aminobutanoic acid, methionine, o-methylserine, t-butylglycine, t-butylalanine, and cyclohexylalanine; group B: aspartic acid, glutamic acid, isoaspartic acid, isoglutamic acid, 2-aminoadipic acid, and 2-aminosuberic acid; group C: asparagine and glutamine; group D: lysine, arginine, ornithine, 2,4-diaminobutanoic acid, and 2,3-diaminopropionic acid; group E: proline, 3-hydroxyproline, and 4-hydroxyproline; group F: serine, threonine, and homoserine; and group G: phenylalanine, tyrosine, and tryptophan.

The identity of an amino acid sequence or a nucleotide sequence can be determined using the Karlin-Altschul algorithm BLAST (PNAS, 1990 (vol. 87) p2264; PNAS, 1993 (vol. 90) p5873). A program called BLASTN or BLASTX based on the BLAST algorithm has been developed (Altschul S F, et al., J Mol Biol, 1990 (vol. 215) p403). In the case of analyzing a nucleotide sequence using BLASTN, parameters are set to, for example, score=100 and wordlength=12. In the case of analyzing an amino acid sequence using BLASTX, parameters are set to, for example, score=50 and wordlength=3. In the case of using the BLAST and Gapped BLAST programs, default parameters of each program are used. Alternatively, the identity between the amino acid sequences of proteins can also be determined according to the expression “(The number of identical amino acid residues/The number of amino acid residues in the whole protein)×100(%)” by aligning the amino acid sequences of the two types of proteins to be compared and counting the number of the same amino acid residues between them.

The correspondence between the human-derived anti-hMPV antibodies found in the present invention and an antigen-binding fragment thereof and SEQ ID NOs of their amino acid sequences in the Sequence Listing is shown in Table 1.

TABLE 1
List of SEQ ID NOs of amino acid sequences
related to various human anti-hMPV antibodies

| | | | Full- | Variable | CDR aa sequence | | |
|---|---|---|---|---|---|---|---|
| | Antibody | H chain/ | length aa | region aa | CDR- | CDR- | CDR- |
| | number | L chain | sequence | sequence | 1 | 2 | 3 |
| 01 | EV046113 | H chain | 1 | 2 | 3 | 4 | 5 |
| | | L chain | 6 | 7 | 8 | 9 | 10 |
| 02 | EV046115b | H chain | 11 | 12 | 13 | 14 | 15 |
| | | L chain | 16 | 17 | 18 | 19 | 20 |
| 03 | EV046116 | H chain | 21 | 22 | 23 | 24 | 25 |
| | | L chain | 26 | 27 | 28 | 29 | 30 |
| 04 | EV046120 | H chain | 31 | 32 | 33 | 34 | 35 |
| | | L chain | 36 | 37 | 38 | 39 | 40 |
| 05 | EV046124 | H chain | 41 | 42 | 43 | 44 | 45 |
| | | L chain | 46 | 47 | 48 | 49 | 50 |
| 06 | EV046130 | H chain | 51 | 52 | 53 | 54 | 55 |
| | | L chain | 56 | 57 | 58 | 59 | 60 |
| 07 | EV046135 | H chain | 61 | 62 | 63 | 64 | 65 |
| | | L chain | 66 | 67 | 68 | 69 | 70 |
| 08 | EV046136 | H chain | 71 | 72 | 73 | 74 | 75 |
| | | L chain | 76 | 77 | 78 | 79 | 80 |
| 09 | EV046141 | H chain | 81 | 82 | 83 | 84 | 85 |
| | | L chain | 86 | 87 | 88 | 89 | 90 |
| 10 | EV046142 | H chain | 91 | 92 | 93 | 94 | 95 |
| | | L chain | 96 | 97 | 98 | 99 | 100 |
| 11 | EV046143 | H chain | 101 | 102 | 103 | 104 | 105 |
| | | L chain | 106 | 107 | 108 | 109 | 110 |
| 12 | EV046147 | H chain | 111 | 112 | 113 | 114 | 115 |
| | | L chain | 116 | 117 | 118 | 119 | 120 |
| 13 | EV046150 | H chain | 121 | 122 | 123 | 124 | 125 |
| | | L chain | 126 | 127 | 128 | 129 | 130 |

(In the table, the number shown in each cell represents each SEQ ID NO.)

Among these obtained antibodies, EV046143 (VH: SEQ ID NO: 102, VL: SEQ ID NO: 107) and EV046150 (VH: SEQ ID NO: 122, VL: SEQ ID NO: 127), whose epitope belonged to the same epitope group and which were very analogous in terms of neutralizing activity and CDR sequences, were analyzed for their sequence identity and consequently found to have approximately 73% identity in the H chain variable regions (SEQ ID NOs: 102 and 122). Approximately 82% identity was confirmed in the amino acid sequences of their L chain variable regions (SEQ ID NOs: 107 and 127). As for the FR regions (FR1+FR2+FR3+FR4) of each chain, 83% identity was confirmed for VH, and approximately 89% identity was confirmed for VL. As for the amino acid sequences of CDRs, 60% (3/5) identity was confirmed in the H chain CDR1 amino acid sequences (SEQ ID NOs: 103 and 123). Approximately 53% (9/17) identity was confirmed in the H chain CDR2 amino acid sequences (SEQ ID NOs: 104 and 124). When the identity was compared between the H chain CDR3 amino acid sequences (SEQ ID NOs: 105 and 125), 37.5% (6/16) identity was observed. In summary, approximately 46% (18/38) identity was confirmed for the whole CDRs of the H chains. On the other hand, when the L chain CDR1 amino acid sequences (SEQ ID NOs: 108 and 128) were compared, approximately 42% (5/12) identity was confirmed. Approximately 87% (6/7) identity was confirmed in the L chain CDR2 amino acid sequences (SEQ ID NOs: 109 and 129). When the L chain CDR3 amino acid sequences (SEQ ID NOs: 120 and 130) were further compared, 75% (6/8) identity was confirmed. In summary, approximately 63% (17/72) identity was confirmed for the whole CDRs of the L chains. Considering these factors together, an antibody having 50% or higher identity to any of the 13 antibodies mentioned above in terms of the whole CDR sequences, or a fragment thereof probably has binding activity and hMPV-neutralizing activity similar to those of the 13 antibodies. Furthermore, an antibody having 60% or higher (preferably 70% or higher, 80% or higher, 90% or higher, 95% or higher, 96% or higher, 97% or higher, 98% or higher, or 99% or higher) identity thereto, in other words, an antibody comprising an amino acid sequence having the deletion, substitution, insertion, or addition of one to several (specifically, 1 to 15, 1 to 10, 1 to 9, 1 to 8, 1 to 7, 1 to 6, 1 to 5, 1 to 4, 1 to 3, 1 to 2, or 1) amino acid residues in the whole three CDR sequences (CDR1, CDR2, and CDR3) of each chain, or the combination of two or more of these mutations, probably has binding activity and neutralizing activity similar to those of the 13 anti-hMPV antibodies.

Thus, the antibody of the present invention or an antigen-binding fragment is preferably an antibody having heavy chain CDR1, CDR2, and CDR3 and light chain CDR1, CDR2, and CDR3 that respectively correspond to 6 amino acid sequences in one combination selected from the combination groups of, for example, SEQ ID NOs: 3 to 5 and 8 to 10, 13 to 15 and 18 to 20, 23 to 25 and 28 to 30, 33 to 35 and 38 to 40, 43 to 45 and 48 to 50, 53 to 55 and 58 to 60, 63 to 65 and 68 to 70, 73 to 75 and 78 to 80, 83 to 85 and 88 to 90, 93 to 95 and 98 to 100, 103 to 105 and 108 to 110, 113 to 115 and 118 to 120, and 123 to 125 and 128 to 130, or an antigen-binding fragment thereof. The CDR sequences, however, may have amino acid sequences derived from the 6 amino acid sequences in one combination selected from the combination groups of SEQ ID NOs: 3 to 5 and 8 to 10, 13 to 15 and 18 to 20, 23 to 25 and 28 to 30, 33 to 35 and 38 to 40, 43 to 45 and 48 to 50, 53 to 55 and 58 to 60, 63 to 65 and 68 to 70, 73 to 75 and 78 to 80, 83 to 85 and 88 to 90, 93 to 95 and 98 to 100, 103 to 105 and 108 to 110, 113 to 115 and 118 to 120, and 123 to 125 and 128 to 130, by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues or 50% or less (preferably 40%, 30%, 20%, or 10% or less) of the number of amino acid residues in the CDR sequences, or the combination of two or more of these mutations, as long as the resulting monoclonal antibody is capable of specifically binding to hMPV F protein and neutralizing its biological activity. Its amino acid sequence except for the CDRs is not particularly limited, and a so-called CDR-grafted antibody having an amino acid sequence, except for CDRs, derived from another antibody, particularly, an antibody of different species is also encompassed by the antibody of the present invention. Of these antibodies, an antibody is more preferred in which the amino acid sequence other than CDRs is also derived from a human. If necessary, its framework regions (FRs) may have the deletion, substitution, insertion, or addition of one to several (specifically, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1) amino acid residues, or the combination of two or more of these mutations.

In some cases, the combination of the 6 CDR sequences contained in the antibody of the present invention may be a combination in which 5 to 1 of the 6 amino acid sequences in one combination selected from the combination groups of SEQ ID NOs: 3 to 5 and 8 to 10, 13 to 15 and 18 to 20, 23 to 25 and 28 to 30, 33 to 35 and 38 to 40, 43 to 45 and 48 to 50, 53 to 55 and 58 to 60, 63 to 65 and 68 to 70, 73 to 75 and 78 to 80, 83 to 85 and 88 to 90, 93 to 95 and 98 to 100, 103 to 105 and 108 to 110, 113 to 115 and 118 to 120, and 123 to 125 and 128 to 130 are replaced with corresponding amino acid sequences in any of the other combinations of the 6 amino acid sequences (e.g., in heavy chain CDR1, CDR2, and CDR3 and light chain CDR1, CDR2, and CDR3 represented by SEQ ID NOs: 3, 4, and 5 and 8, 9, and 10, respectively, heavy chain CDR2 (SEQ ID NO: 4) and CDR3 (SEQ ID NO: 5) are replaced with heavy chain CDR2 (SEQ ID NO: 14) and CDR3 (SEQ ID NO: 15), respectively, in the combination of SEQ ID NOs: 13 to 15 and 18 to 20), as long as the resulting monoclonal antibody is capable of specifically binding to hMPV F protein and neutralizing its biological activity. Such an antibody is also included in the scope of the present invention.

In a preferred alternative embodiment, the anti-hMPV antibody according to the present invention is an antibody containing (a) a heavy chain variable region (VH) selected from the group consisting of; the amino acid sequences of SEQ ID NOs: 2, 12, 22, 32, 42, 52, 62, 72, 82, 92, 102, 112, and 122; amino acid sequences derived from any of the amino acid sequences of SEQ ID NOs: 2, 12, 22, 32, 42, 52, 62, 72, 82, 92, 102, 112, and 122 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; and amino acid sequences having 60% or higher (preferably 70% or higher, 80% or higher, 90% or higher, 95% or higher, 96% or higher, 97%, 98% or higher, or 99% or higher) identity to any of the amino acid sequences of SEQ ID NOs: 2, 12, 22, 32, 42, 52, 62, 72, 82, 92, 102, 112, and 122, and (b) a light chain variable region (VL) selected from the group consisting of: the amino acid sequences of SEQ ID NOs: 7, 17, 27, 37, 47, 57, 67, 77, 87, 97, 107, 117, and 127; amino acid sequences derived from any of the amino acid sequences of SEQ ID NOs: 7, 17, 27, 37, 47, 57, 67, 77, 87, 97, 107, 117, and 127 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; and amino acid sequences having 60% or higher (preferably 70% or higher, 80% or higher, 90% or higher, 95% or higher, 96% or higher, 97%, 98% or higher, or 99% or higher) identity to any of the amino acid sequences of SEQ ID NOs: 7, 17, 27, 37, 47, 57, 67, 77, 87, 97, 107, 117, and 127, or an antigen-binding fragment thereof.

In a further preferred alternative embodiment, the anti-hMPV antibody according to the present invention contains (a-1) a heavy chain variable region (VH) represented by the amino acid sequence of SEQ ID NO: 2; an amino acid sequence derived from the amino acid sequence of SEQ ID NO: 2 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 2, and (b-1) a light chain variable region (VL) represented by the amino acid sequence of SEQ ID NO: 7; an amino acid sequence derived from the amino acid sequence of SEQ ID NO: 7 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 7.

In a preferred alternative embodiment, the anti-hMPV antibody contains (a-2) a heavy chain variable region (VH) represented by the amino acid sequence of SEQ ID NO: 12; an amino acid sequence derived from the amino acid sequence of SEQ ID NO: 12 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 12, and (b-2) a light chain variable region (VL) represented by the amino acid sequence of SEQ ID NO: 17; an amino acid sequence derived from the amino acid sequence of SEQ ID NO: 17 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 17.

In a preferred alternative embodiment, the anti-hMPV antibody contains (a-3) a heavy chain variable region (VH) represented by the amino acid sequence of SEQ ID NO: 22; an amino acid sequence derived from the amino acid sequence of SEQ ID NO: 22 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 22, and (b-3) a light chain variable region (VL) represented by the amino acid sequence of SEQ ID NO: 27; an amino acid sequence derived from the amino acid sequence of SEQ ID NO: 27 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 27.

In a preferred alternative embodiment, the anti-hMPV antibody according to the present invention contains (a-4) a heavy chain variable region (VH) represented by the amino acid sequence of SEQ ID NO: 33; an amino acid sequence derived from the amino acid sequence of SEQ ID NO: 33 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 33, and (b-4) a light chain variable region (VL) represented by the amino acid sequence of SEQ ID NO: 38; an amino acid sequence derived from the amino acid sequence of SEQ ID NO: 38 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 38.

In a preferred alternative embodiment, the anti-hMPV antibody contains (a-5) a heavy chain variable region (VH) represented by the amino acid sequence of SEQ ID NO: 43; an amino acid sequence derived from the amino acid sequence of SEQ ID NO: 43 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 43, and (b-5) a light chain variable region (VL) represented by the amino acid sequence of SEQ ID NO: 47; an amino acid sequence derived from the amino acid sequence of SEQ ID NO: 47 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 47.

In a preferred alternative embodiment, the anti-hMPV antibody contains (a-6) a heavy chain variable region (VH) represented by the amino acid sequence of SEQ ID NO: 52; an amino acid sequence derived from the amino acid sequence of SEQ ID NO: 57 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 57, and (b-6) a light chain variable region (VL) represented by the amino acid sequence of SEQ ID NO: 62; an amino acid sequence derived from the amino acid sequence of SEQ ID NO: 62 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 62.

In a preferred alternative embodiment, the anti-hMPV antibody according to the present invention contains (a-7) a heavy chain variable region (VH) represented by the amino acid sequence of SEQ ID NO: 62; an amino acid sequence derived from the amino acid sequence of SEQ ID NO: 62 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 62, and (b-7) a light chain variable region (VL) represented by the amino acid sequence of SEQ ID NO: 67; an amino acid sequence derived from the amino acid sequence of SEQ ID NO: 67 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 67.

In a preferred alternative embodiment, the anti-hMPV antibody contains (a-8) a heavy chain variable region (VH) represented by the amino acid sequence of SEQ ID NO: 72; an amino acid sequence derived from the amino acid sequence of SEQ ID NO: 72 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 72, and (b-8) a light chain variable region (VL) represented by the amino acid sequence of SEQ ID NO: 77; an amino acid sequence derived from the amino acid sequence of SEQ ID NO: 77 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 77.

In a preferred alternative embodiment, the anti-hMPV antibody contains (a-9) a heavy chain variable region (VH) represented by the amino acid sequence of SEQ ID NO: 82; an amino acid sequence derived from the amino acid sequence of SEQ ID NO: 82 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 82, and (b-9) a light chain variable region (VL) represented by the amino acid sequence of SEQ ID NO: 87; an amino acid sequence derived from the amino acid sequence of SEQ ID NO: 87 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 87.

In a preferred alternative embodiment, the anti-hMPV antibody according to the present invention contains (a-10) a heavy chain variable region (VH) represented by the amino acid sequence of SEQ ID NO: 92; an amino acid sequence derived from the amino acid sequence of SEQ ID NO: 92 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 92, and (b-10) a light chain variable region (VL) represented by the amino acid sequence of SEQ ID NO: 97; an amino acid sequence derived from the amino acid sequence of SEQ ID NO: 97 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 97.

In a preferred alternative embodiment, the anti-hMPV antibody contains (a-11) a heavy chain variable region (VH) represented by the amino acid sequence of SEQ ID NO: 102; an amino acid sequence derived from the amino acid sequence of SEQ ID NO: 102 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 102, and (b-11) a light chain variable region (VL) represented by the amino acid sequence of SEQ ID NO: 107; an amino acid sequence derived from the amino acid sequence of SEQ ID NO: 107 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 107.

In a preferred alternative embodiment, the anti-hMPV antibody contains (a-12) a heavy chain variable region (VH) represented by the amino acid sequence of SEQ ID NO: 112; an amino acid sequence derived from the amino acid sequence of SEQ ID NO: 112 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 112, and (b-12) a light chain variable region (VL) represented by the amino acid sequence of SEQ ID NO: 117; an amino acid sequence derived from the amino acid sequence of SEQ ID NO: 117 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 117.

In a preferred alternative embodiment, the anti-hMPV antibody contains (a-13) a heavy chain variable region (VH) represented by the amino acid sequence of SEQ ID NO: 122; an amino acid sequence derived from the amino acid sequence of SEQ ID NO: 122 by the deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 60% or higher (preferably 70% or higher, 80% or higher, 90% or higher, 95% or higher, 96% or higher, 97% or higher, 98% or higher, or 99% or higher) identity to the amino acid sequence of SEQ ID NO: 122, and (b-13) a light chain variable region (VL) represented by the amino acid sequence of SEQ ID NO: 127; an amino acid sequence derived from the amino acid sequence of SEQ ID NO: 127 by the deletion, substitution, insertion, or addition of one to several (specific numbers are the same as above) amino acid residues, or the combination of two or more of these mutations; or an amino acid sequence having 60% or higher (specific % is the same as above) identity to the amino acid sequence of SEQ ID NO: 127.

A method known in the art can be used as a method for preparing these antibodies (Riechmann L, et al., Reshaping human antibodies for therapy. Nature, 332:323-327, 1988). In the present invention, a completely human antibody is preferred, as a matter of course.

### 3. Nucleic Acid Encoding Antibody According to the Present Invention, Etc.

In another embodiment, the present invention provides a nucleic acid (nucleotide) encoding the anti-hMPV antibody capable of specifically binding to hMPV F protein and neutralizing its biological activity, or an antigen-binding fragment thereof. A nucleic acid encoding an amino acid sequence selected from the group consisting of SEQ ID NOs: 4 to 15, 18 to 29, and 32 to 43, and an isolated nucleic acid having high identity to the nucleic acid are also included in the scope of the present invention. In this context, the term “having high identity” means sequence identity to an extent that permits hybridization under high stringent conditions to the predetermined nucleic acid sequence and means having, for example, 60%, 70%, 80%, 90%, or 95% or higher identity. The present invention provides an isolated nucleic acid selected from nucleic acids hybridizing thereto under high stringent conditions. Preferably, the nucleic acid is DNA or RNA, more preferably DNA.

The “high stringent conditions” are conditions involving, for example, 5×SSC, 5×Denhardt's solution, 0.5% SDS, 50% formamide, and 50° C. (See e.g., J. Sambrook et al., Molecular Cloning, A Laboratory Manual 2nd ed., Cold Spring Harbor Laboratory Press (1989), particularly, 11.45 “Conditions for Hybridization of Oligonucleotide Probes”). Under these conditions, it can be expected that a polynucleotide (e.g., DNA) having high identity is more efficiently obtained at a higher temperature. However, possible factors that influence the stringency of hybridization are a plurality of factors such as temperatures, probe concentrations, probe lengths, ionic strengths, times, and salt concentrations. Those skilled in the art can achieve similar stringency by appropriately selecting these factors.

The nucleic acid hybridizing under high stringent conditions includes a nucleic acid having, for example, 70% or higher, 80% or higher, 90% or higher, 95% or higher, 97% or higher, or 99% or higher identity to the nucleic acid encoding the amino acid sequence.

The identity of a nucleotide sequence can be determined by use of the aforementioned identity search algorithm or the like (Proc. Natl. Acad. Sci. USA 872264-2268, 1990; and Proc Natl Acad Sci USA 90: 5873, 1993).

The nucleic acid encoding the anti-hMPV antibody of the present invention is preferably a DNA encoding the H chain or L chain of an antibody comprising CDR sequences in any one of the combinations of 26 CDR sequences of SEQ ID NOs: 3 to 5, 8 to 10, 13 to 15, 18 to 20, 23 to 25, 28 to 30, 33 to 35, 38 to 40, 43 to 45, 48 to 50, 53 to 55, 58 to 60, 63 to 65, 68 to 70, 73 to 75, 78 to 80, 83 to 85, 88 to 90, 93 to 95, 98 to 100, 103 to 105, 108 to 110, 113 to 115, 118 to 120, 123 to 125, and 128 to 130, or an antigen-binding fragment thereof, more preferably a DNA encoding the H chain or L chain of an antibody comprising any VH or VL selected from SEQ ID NOs: 2, 7, 12, 17, 22, 27, 32, 37, 42, 47, 52, 57, 62, 67, 72, 77, 82, 87, 92, 97, 102, 107, 112, 117, 122, and 127, or an antigen-binding fragment thereof, or an isolated nucleic acid hybridizing under high stringent conditions to any of these nucleic acids (DNAs).

The nucleic acid is further preferably a nucleic acid encoding the amino acid sequence of SEQ ID NO: 1, 6, 11, 16, 21, 26, 31, 36, 41, 46, 51, 56, 61, 66, 71, 76, 81, 86, 91, 96, 101, 106, 111, 116, 121, or 126, or an isolated nucleic acid hybridizing under high stringent conditions to any of these nucleic acids.

### 4. Vector, Host Cell, and Antibody Preparation Method According to the Present Invention

The present invention also relates to a vector comprising the nucleic acid incorporated therein and a host cell comprising the vector introduced thereinto, and a method for preparing the antibody using the vector and the host cell.

The antibody of the present invention can also be prepared as a recombinant human antibody by use of a method known in the art (see e.g., Nature, 312: 643, 1984; and Nature, 321: 522, 1986). The antibody of the present invention can be prepared, for example, by culturing the host cell having the vector according to the present invention incorporated therein and purifying the produced antibody from the culture supernatant or the like. More specifically, VH- and VL-encoding cDNAs can be respectively inserted to expression vectors for animal cells containing human antibody CH- and/or human antibody CL-encoding genes prepared from the same cell or another human cell to construct human antibody expression vectors, which are transferred to animal cells so that the antibody is produced by expression.

The vector to which the nucleic acid encoding VH or VL of the antibody of the present invention is incorporated is not necessarily limited and is preferably a vector that is routinely used for the expression of protein genes or the like and is particularly compatible with the expression of antibody genes, or a vector for high expression. Preferred examples thereof include vectors containing EF promoter and/or CMV enhancer. Usually, expression vectors respectively having VH- or VL-encoding nucleic acids incorporated therein are prepared, and host cells are cotransfected with the expression vectors. Alternatively, these nucleic acids may be incorporated in a single expression vector.

The host cell to which the expression vector is transferred is not necessarily limited and is preferably a cell that is routinely used for the expression of protein genes or the like and is particularly compatible with the expression of antibody genes. Examples thereof include bacteria (E. coli, etc.), Actinomycetes, yeasts, insect cells (SF9, etc.), and mammalian cells (COS-1, CHO, myeloma cells, etc.).

For the industrial production of the recombinant antibody, a recombinant animal cell line, for example, a CHO cell line, stably highly producing the antibody is generally used. The preparation and cloning of such a recombinant cell line, gene amplification for high expression, and screening can be carried out by use of methods known in the art (see e.g., Omasa T.: J. Biosci. Bioeng., 94, 600-605, 2002).

The present invention includes an antibody composed of two heavy chains and two light chains as well as an antigen-binding fragment of the antibody of the present invention. The antigen-binding fragment includes, for example, Fab (Fragment of antigen binding), Fab′, F(ab′)2, and single chain Fv (scFv) and disulfide stabilized Fv (dsFv) as active fragments of the antibody linked via a linker or the like. Examples of peptides comprising the active fragments of the antibody include peptides containing CDRs. These fragments can be produced by a method known in the art such as a method of treating the antibody of the present invention with an appropriate protease or a gene recombination technique.

The antibody can be purified by use of purification means known in the art such as salting out, gel filtration, ion-exchange chromatography, or affinity chromatography.

Alternatively, according to a recently developed phage display antibody technique which involves expressing a recombinant antibody on phage surface by use of a genetic engineering technique, VH and VL genes may be artificially shuffled, and diversified scFv (single chain Fragment of variable region) antibodies can be expressed as phage-fused proteins to obtain specific antibodies. This technique can circumvent immunity and is highly appreciated as a humanized antibody preparation technique as an alternative for the cell fusion method. Any specific antibody or antigen-binding fragment thereof prepared by use of this technique with reference to the amino acid sequences of SEQ ID NOs: 2 to 6, 8 to 12, 14 to 18, and 20 to 24 described herein is included in the technical scope of the present invention.

Alternatively, an antibody obtained by the application of Potelligent technique drastically improving the ADCC activity of an antibody by the modification of the sugar chain moiety of the antibody prepared by a recently developed technique, to the antibody of the present invention (see Clin. Cancer Res., 10, 6248-6255 (2004)), and an antibody obtained by the application of Complegent technique improving CDC activity, to the antibody of the present invention (see Glycobiology, 17, 104-118 (2007)) are also included in the technical scope of the present invention. Likewise, an antibody prepared by a recently developed method for modifying ADCC activity (WO2007/039682 and WO2007/100083) or CDC activity (WO2007/011041 and WO2011/091078) by the modification of a partial amino acid sequence in the constant region of the antibody is also included in the technical scope of the present invention.

Furthermore, any antibody or antigen-binding fragment thereof obtained by the application of a partial Fc region substitution technique (see WO2006/071877) performed in order to impart protease resistance ability to the antibody and renders the antibody orally administrable is included in the technical scope of the present invention.

As an antibody preparation approach, a polyclonal antibody or a monoclonal antibody is usually obtained by use of a laboratory animal such as a mouse, a rabbit, or a goat. Since the antibody thus obtained has a sequence characteristic of the animal species used, the antibody administered directly to a human may be recognized as foreign matter by the human immune system to cause human anti-animal antibody response (i.e., to yield an antibody against the antibody).

The anti-hMPV monoclonal antibody according to the present invention or an antigen-binding fragment thereof can be obtained from antibody-producing cells derived from the blood of a healthy person or the like. In this case, the antibody is a completely human antibody. This completely human antibody probably neither has immunogenicity nor causes immune response even if administered as an antibody drug to a human body.

Particularly, the monoclonal antibody of the present invention against hMPV F protein has higher neutralizing ability than that of the conventional anti-hMPV antibodies and as such, can be expected to produce equivalent therapeutic effects at a lower dose.

### 5. Pharmaceutical Composition Containing Antibody According to the Present Invention

The present invention further provides a pharmaceutical composition for the prevention or treatment of a respiratory disease caused by hMPV, comprising the antibody or an antigen-binding site thereof and a pharmaceutically acceptable carrier.

Particularly, the anti-hMPV antibody according to the present invention or an antigen-binding fragment thereof has, through specific binding to hMPV F protein, higher neutralizing ability than that of the conventional anti-hMPV F protein antibodies and as such, is useful as a preventive or therapeutic drug for a disease involving the hMPV.

The “pharmaceutically acceptable carrier” used herein includes any or every biologically compatible solvent, dispersion medium, coating, tonicity agent, and absorption-delaying agent, etc.

Examples of the pharmaceutically acceptable carrier include one or more carriers such as water, a salt solution, phosphate-buffered saline, dextrose, glycerol, and ethanol, and combinations thereof. For use as an injection or the like, the composition preferably contains a pH adjuster or a tonicity agent, for example, a sugar, a polyalcohol (mannitol, sorbitol, etc.), or sodium chloride. The pharmaceutically acceptable carrier can further include a small amount of an auxiliary substance that enhances the conservation or effectiveness of the antibody or a moiety of the antibody, such as a wetting agent, an emulsifier, an antiseptic, a buffer, or a stabilizer.

The composition of the present invention can be prepared in various dosage forms. Such a composition includes liquid, semisolid, and solid dosage forms, for example, solutions (e.g., injectable and transfusable solutions), dispersions, suspensions, tablets, capsules, troches, pills, powders, liposomes, and suppositories. Preferred forms differ depending on the intended mode of administration and a case to which treatment is applied. The composition is generally preferably in the form of an injectable or transfusable solution, as with, for example, compositions similar to those used for the passive immunization of humans with other antibodies. The preferred mode of administration is parenteral administration (e.g., intravenous, subcutaneous, intraperitoneal, or intramuscular administration). In a preferred embodiment, the antibody is administered through intravenous transfusion or intravenous injection. In another preferred embodiment, the antibody is administered through intramuscular injection or subcutaneous injection.

The antibody of the present invention or the antibody fragment can be incorporated into a pharmaceutical composition suitable for parenteral administration. When one type of antibody or moiety of the antibody is used, the antibody or the moiety is preferably prepared as an injectable preparation containing 0.1 to 250 mg/mL of the antibody (moiety). On the other hand, when plural types of antibodies are used as a mixture, these antibodies are preferably prepared as an injectable preparation containing 0.001 to 100 mg/mL of each antibody. The mixing ratio of the plural types of antibodies can be appropriately set.

Exemplary injectable preparations will be described below. However, the preparation of the present invention is not limited thereto as long as the preparation is preferred as an injection of the antibody drug of the present invention. The preparation can be constituted by, for example, a flint or amber vial, an ampule, or a prefilled syringe containing an active ingredient dissolved in a liquid or a freeze-dried active ingredient. The buffer can be L-histidine (1 to 50 mM) having a pH of 5.0 to 7.0 (optimally pH 6.0), optimally 5 to 10 mM L-histidine. Other appropriate buffers include, but are not limited to, sodium succinate, sodium citrate, sodium phosphate, and potassium phosphate. Sodium chloride can be used in order to change the osmotic pressure of a solution having a concentration of 0 to 300 mM (optimally 150 mM for the liquid dosage form,). The freeze-dried dosage form can contain a cryoprotectant, mainly, 0 to 10% (optimally 0.5 to 5.0%) sucrose. Other appropriate cryoprotectants include mannitol, trehalose, and lactose. The freeze-dried dosage form can contain an expander, mainly, 1 to 10% (optimally 2 to 4%) mannitol. In both of the liquid and freeze-dried dosage forms, a stabilizer, mainly, 1 to 50 mM (optimally 5 to 10 mM) L-methionine can be used. Other appropriate stabilizers include glycine, arginine, and polysorbate 80, etc. In the case of polysorbate 80, 0 to 0.05% (optimally 0.005 to 0.01%) can be contained therein. Other surfactants include, but are not limited to, polysorbate 20 and BRU surfactants.

In general, the pharmaceutical composition of the present invention must be sterile or stable under production and preservation conditions. This composition can be formulated as a solution, a microemulsion, a dispersion, a liposome, or other ordered structures suitable for high drug concentrations. The sterile injectable solution can be prepared by mixing a necessary amount of an active compound (i.e., the antibody or a moiety of the antibody), if necessary together with one or combination of the aforementioned components, into an appropriate solvent, followed by filtration sterilization. In general, the active compound is mixed with a sterile vehicle containing a basic dispersion medium and other necessary components selected from those listed above to prepare a dispersion. Preferred methods for preparing a sterile powder preparation for preparing the sterile injectable solution are the vacuum freeze drying and spray drying of the sterile filtrate thereof mentioned above. As a result, a composition comprising a powder of the active ingredient as well as arbitrary other desired components is obtained. The adequate flowability of the solution can be maintained, for example, by using coating with lecithin or the like, by maintaining a necessary particle size in the case of the dispersion, or by using a surfactant. The long-term absorption of the injectable composition can be achieved by means of absorption-delaying agent, for example, monostearate or gelatin, contained in the composition.

### 6. Process of Obtaining Anti-hMPV Antibody According to the Present Invention and Antigen-Binding Fragment Thereof

Next, the processes by which the anti-hMPV monoclonal antibody according to the present invention and an antigen-binding fragment thereof were obtained will be described. However, the approaches of obtaining the antibody according to the present invention, etc., are not limited by the description below. As mentioned above, changes or modifications usually performed in the art can be made therein, as a matter of course.

The anti-hMPV antibody according to the present invention and an antigen-binding fragment thereof can be obtained by: separating a cell clone producing the antibody through various steps from the blood of a human; and subjecting the antibody obtained from the culture supernatant of the antibody-producing cell clone to affinity purification.

1) Separation of Cell Clone Producing Completely Human Antibody Against F Proteins of hMPV

B lymphocytes are separated from the blood of a human, and the growth of the B lymphocytes is induced. The method for inducing the growth is known in the art per se and can be carried out by, for example, a transformation method (D. Kozbor et al.) using “Epstein-Barr virus (EB virus)” (hereinafter, referred to as EBV), which is a factor triggering cancer.

Specifically, the B lymphocytes are infected by EBV to induce its growth. The cells that have grown are used as an antibody-producing cell library.

2) Recovery of Monoclonal Antibody from Antibody-Producing Cell Library

The method for recovering monoclonal antibodies from the cells that have grown by induction can be carried out by a well-known method routinely used in the preparation of monoclonal antibodies.

The antibody-producing cell library is screened for a lymphocyte clone producing an antibody binding to hMPV F protein. The antibody is isolated from the culture supernatant thereof. Specifically, a cell population (clone) producing the antibody binding to hMPV F protein is selected from the antibody-producing cell library by a limiting dilution method.

The clone binding to hMPV F protein can be detected by use of ELISA with the F protein as an antigen and ELISA using a labeled mouse anti-human IgG antibody. An alternative method that may be used involves introducing an F protein expression vector into cells, and after expression of the F protein, immobilizing the cells onto a screening plate, which is then used in the detection of the anti-hMPV antibody (cell fluorescent immunostaining screening method).

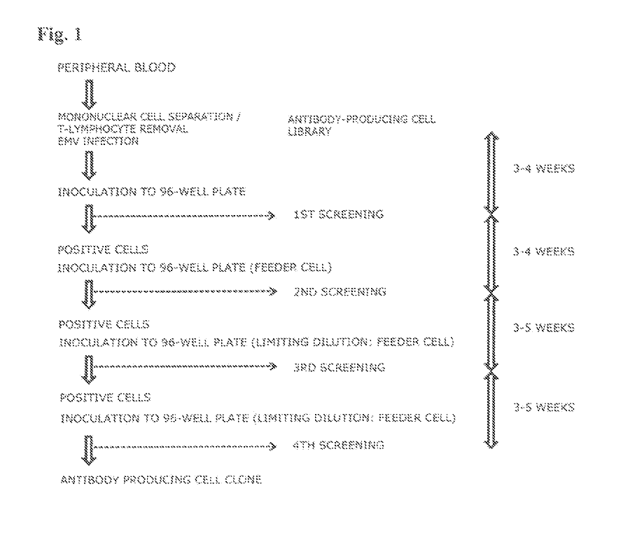

The selected antibody-positive cell population can be cultured and repeatedly screened to obtain a cell population (clone) producing only the antibody of interest. A flowchart representing these steps up to the separation of the antibody-producing cell clone is shown in FIG. 1.

### 3) Affinity Purification Using Protein A or G

For the purification of the anti-hMPV antibody, the selected cells can be allowed to grow in a dish, a roller bottle, a 2-L spinner flask, or a different culture system.

The obtained culture supernatant can be filtered, concentrated, and then subjected to affinity chromatography using protein A or protein G-Sepharose (GE Healthcare Japan Corp.) or the like to purify the protein. The buffer solution is replaced with PBS, and the concentration can be determined by OD280 or, preferably, nephelometer analysis. The isotype can be examined by a method specific for the isotype antigen. The anti-hMPV antibody thus obtained is a completely human antibody prepared from B lymphocytes sensitized in the human body and is therefore substantially unlike to cause immune response.

Another feature of this approach is that the EB virus having the activity of inducing the growth of B lymphocytes by infection is used in the preparation of the antibody-producing cell clone.

The EB virus method has the advantages that: a natural antibody produced in a human body can be prepared; and an antibody having high affinity can be obtained. For example, a human antibody against a certain kind of virus (e.g., human CMV) has been found to have approximately 10 to 100 times higher affinity than that of an antibody prepared from an artificially immunized mouse. The B lymphocyte population that has grown by EB virus infection serves as a library of antibody-producing cells. From this library, a particular antibody-producing cell clone is separated to obtain a human antibody.

### 7. Process of Evaluating Activity In Vitro

The biological properties of the antibody or an antibody composition can be evaluated by testing the ability of the antibody to suppress in vitro the biological activity of the hMPV F protein. The in vitro assay method for the antibody includes a binding assay method such as ELISA, and a neutralization assay method, etc.

### 1) Binding Activity

The term “specifically binding” or “specific binding” used herein refers to the recognition of the predetermined antigen so as to bind thereto. The binding affinity of the antibody for the hMPV F protein can be measured according to a method known in the art. For example, the binding affinity can be measured against F protein immobilized on a chip by use of a protein interaction analysis apparatus such as Biacore T200®. The binding affinity (K_{D} value) is indicated by the ratio of Kd (dissociation constant) obtained by this measurement method to Ka (association constant) (K_{D}=Kd/Ka). The binding affinity (KD value) of the human anti-hMPV antibody according to the present invention or an antigen-binding fragment thereof against JPS02-76 (hMPV_type B1) F protein is preferably 1×10⁻⁸ M or lower, more preferably 1×10⁻⁹ M or lower, further preferably 6×10⁻¹⁰ M or lower.

### 2) In Vitro Neutralizing Activity

The terms such as “neutralization”, “inhibitory effect”, “inhibition”, “suppression”, and “capable of inhibiting” used herein mean that biological activity attributed to the antigen (hMPV) is reduced by approximately 5 to 100%, preferably 10 to 100%, more preferably 20 to 100%, more preferably 30 to 100%, more preferably 40 to 100%, more preferably 50 to 100%, more preferably 60 to 100%, more preferably 70 to 100%, further preferably 80 to 100%.

The in vitro neutralizing ability of the anti-hMPV antibody can be evaluated using typical strains of four hMPV subgroups (types A1, A2, B1, and B2) (J. Virol. 2008 p. 8942-8946). The hMPV strains used are, for example, a JPS03-180 strain of type A1, a JPS03-178 strain of type A2, a JPS02-76 strain of type B1, and a JPS05-21 strain of type B2. The ability of each strain to infect LLC-MK2 cells can be examined in the presence of the anti-hMPV antibody to evaluate the neutralizing ability of the antibody.

For reference, among the previously reported anti-hMPV antibodies, an antibody mAb338 (WO2006/110214), albeit derived from a mouse, has been reported to have high neutralizing activity against all of the hMPV types A1, A2, B1, and B2, and a human antibody HMB3210 (WO2013/140247) exhibits high neutralizing activity against both RSV and hMPV. These antibodies were both used as positive controls.

The anti-hMPV antibody according to the present invention or an antigen-binding fragment thereof has 50% infection inhibitory activity (IC50) at preferably approximately 2.3 μg/mL (approximately 15 nM) or lower, more preferably approximately 1 μg/mL (approximately 6.7 nM) or lower, further preferably approximately 0.6 μg/mL (approximately 4 nM) or lower, most preferably approximately 0.3 μg/mL (approximately 2 nM) or lower, against all of the strains JPS03-180 (type A1), JPS03-178 (type A2), JPS02-76 (type B1), and JPS05-21 (type B2) in an evaluation system for the ability of hMPV to infect LLC-MK2 cells. Alternatively, the anti-hMPV antibody according to the present invention or an antigen-binding fragment thereof has 90% infection inhibitory activity (IC90) at preferably approximately 3.5 μg/mL (approximately 23 nM) or lower, more preferably approximately 2 μg/mL (approximately 13.3 nM) or lower, further preferably approximately 1 μg/mL (approximately 6.7 nM) or lower, most preferably approximately 0.7 μg/mL (approximately 4.7 nM) or lower, against all of the strains JPS03-180 (type A1), JPS03-178 (type A2), JPS02-76 (type B1), and JPS05-21 (type B2).

Alternatively, a human antibody having 50% infection inhibitory activity (IC50) at approximately 2 μg/mL (approximately 13.3 nM) or lower, preferably approximately 1 μg/mL (approximately 6.7 nM) or lower, more preferably approximately 0.5 μg/mL (approximately 3.3 nM) or lower, further preferably approximately 0.2 μg/mL (approximately 1.33 nM) or lower, still further preferably approximately 0.1 μg/mL (approximately 0.67 nM) or lower, most preferably approximately 0.03 μg/mL (approximately 0.2 nM) or lower, in an evaluation system using JPS03-178 (type A2), or an antigen-binding fragment thereof is included in the scope of the present invention.

Alternatively, a human antibody having 90% infection inhibitory activity (IC90) at approximately 6 μg/mL (approximately 40 nM) or lower, preferably approximately 1 μg/mL (approximately 6.7 nM) or lower, more preferably approximately 0.2 μg/mL (approximately 1.33 nM) or lower, further preferably approximately 0.1 μg/mL (approximately 0.67 nM) or lower, in an evaluation system using JPS03-178 (type A2), or an antigen-binding fragment thereof is included in the scope of the present invention.

### 3) Cell-to-Cell Spread Inhibitory Activity

The cell-to-cell spread of hMPV requires the cleavage of FP. LLC-MK2 cells or VERO cells, which serve as host cells for in vitro hMPV infection, lack the activity of cleaving FP. Accordingly, membrane-type serine protease (TMPRSS2) having the activity of cleaving FP is introduced into LLC-MK2 cells to prepare host cells stably expressing TMPRSS2. The obtained cells stably expressing TMPRSS2 are infected by hMPV. In this system, the cell-to-cell spread inhibitory activity of the anti-hMPV antibody can be evaluated. When the cell-to-cell spread inhibitory activity of the anti-hMPV antibody is evaluated in a system in which the host cells are infected by a JPS02-76 strain of hMPV type B1, a human antibody having 50% cell-to-cell spread inhibitory activity (IC50) at approximately 0.2 μg/mL (approximately 1.33 nM) or lower, preferably approximately 0.1 μg/mL (approximately 0.67 nM) or lower, more preferably approximately 0.05 μg/mL (approximately 0.33 nM) or lower, further preferably approximately 0.02 μg/mL (approximately 0.133 nM) or lower, or an antigen-binding fragment thereof is included in the scope the present invention.

### 8. Epitope Grouping

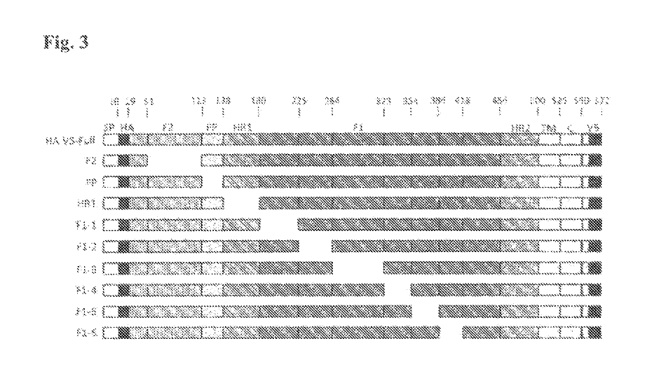

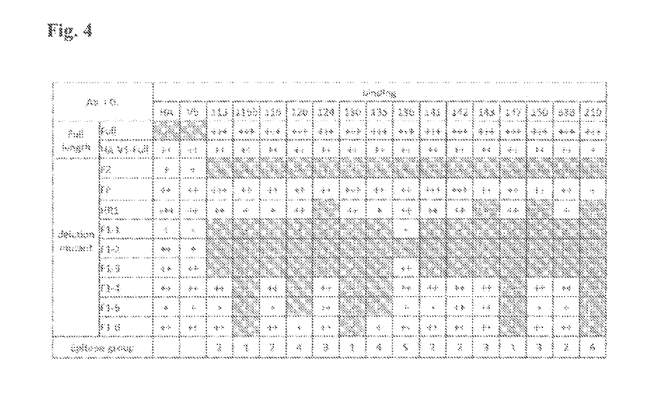

The difference in epitope among anti-hMPV antibodies can be evaluated by the competition assay among the antibodies and the examination of the binding activity of each antibody against cells expressing a partially deleted peptide of the F protein (see the paragraph “12. Epitope mapping” in Examples and FIGS. 3 and 4). As a result, the anti-hMPV antibody of the present invention can be classified into any of 5 epitope groups (Group 1, Group 2, Group 3, Group 4, and Group 5). Specifically, Group 1 (group of antibodies binding to FP and HR1 deletion mutants, but not binding to F2, F1-1, F1-2, F1-3, F1-4, F1-5, and F1-6 deletion mutants) includes EV046115b, EV046130, and EV046147. Group 2 (group of antibodies binding to FP, HR1, F1-4, F1-5, and F1-6 deletion mutants, but not binding to F2, F1-1, F1-2, and F1-3 deletion mutants) includes mAb338, EV046113, EV046116, EV046141, and EV046142. Group 3 (group of antibodies binding to FP, F1-4, F1-5, and F1-6 deletion mutants, but not binding to F2, HR1, F1-1, F1-2, and F1-3 deletion mutants) includes EV046124, EV046143, and EV046150. Group 4 (group of antibodies binding to FP, HR1, and F1-6 deletion mutants, but not binding to F2, F1-1, F1-2, F1-3, F1-4, and F1-5 deletion mutants) includes EV046120, and EV046135. Group 5 (group of antibodies binding to FP, HR1, F1-1, F1-3, F1-4, F1-5, and F1-6 deletion mutants, but not binding to F2 and F1-2 deletion mutants) includes EV046136. Thus, the anti-hMPV antibody of the present invention is an antibody binding to hMPV F protein or an antigen-binding fragment thereof and is preferably an antibody binding to any of the epitope groups defined by Group 1, Group 2, Group 3, Group 4, and Group 5, or an antigen-binding fragment thereof, more preferably an antibody binding to any of the epitope groups defined by Group 2, Group 3, Group 4, and Group 5, or an antigen-binding fragment thereof, further preferably an antibody binding to any of the epitope groups defined by Group 3, Group 4, and Group 5, or an antigen-binding fragment thereof, most preferably an antibody binding to any of the epitope group defined by Group 3, or an antigen-binding fragment thereof.

For the epitope grouping, among the previously reported anti-hMPV antibodies, a mouse antibody mAb338 (WO2006/110214) having high neutralizing activity against all of the hMPV strains A1, A2, B1, and B2 and already reported to be analyzed for its epitope (a.a. 238-245 of F protein), and a human antibody HMB3210 (WO2013/140247) binding both to the F proteins of hMPV and RSV were both used as controls for measurement.

Hereinafter, the present invention will be described further specifically with reference to Examples. However, the present invention is not intended to be limited by these Examples by any means. For the procedures used in these Examples, see Molecular Cloning: A Laboratory Manual (Third Edition) (Sambrook et al., Cold Spring Harbour Laboratory Press, 2001), unless otherwise specified.

### Examples

### 1. Separation of Cell Clone Producing Completely Human Antibody Against hMPV

A flowchart for the separation of an antibody-producing cell clone is shown in FIG. 1.

B lymphocytes were separated from human peripheral blood and infected by EBV. The infected cells were inoculated to a 96-well plate, cultured for 3 to 4 weeks, and then primarily screened for anti-hMPV antibodies in the culture supernatant. The cells in each well confirmed to contain the produced anti-hMPV antibodies were inoculated to a fresh 96-well plate. After culture for 3 to 4 weeks, the cells were secondarily screened for anti-hMPV antibodies. The cells in each antibody-positive well were inoculated at 1 to 30 cells/well to a fresh 96-well plate. After culture for 3 to 5 weeks, the cells were tertiarily screened for anti-hMPV antibodies. As a result of this cloning operation involving the limiting dilution culture method, etc., a cell clone producing each antibody of interest was obtained.

### 2. Cell Fluorescent Immunostaining Screening of Anti-hMPV Antibody

The screening targeted antibodies against fusion protein (FP), which is a main neutralization target in hMPV. RNA was extracted from hMPV (JPS02-76), and cDNA was synthesized using random decamers. The full-length FP gene was amplified by PCR and cloned into an expression vector. The nucleotide sequence of the cloned FP gene and an amino acid sequence encoded thereby are shown in SEQ ID NOs: 131 and 132, respectively. The cloned FP gene differs by 2 bases from the original sequence registered in the gene bank (GenBank Accession No. AY530089) and resulted in one amino acid substitution.

CHO-K1 cells were transfected with the FP expression vector. After 16 hours, the cells were inoculated again to a 96-well plate or a 384-well plate. This transfection was carried out using Lipofectamine LTX (Invitrogen Corp.) and Plus reagent (Invitrogen Corp.) under conditions recommended by the manufacturer. The CHO-K1 cells then cultured for 24 hours were fixed using 4% paraformaldehyde and 0.1 M phosphate buffer (pH 7.4). Next, the cells were washed with PBS-0099PBS(−) and 0.1% Tween 20 and permeabilized using 1×PBS(−) and 0.2% Triton X-100. The prepared plate was used as an anti-hMPV antibody screening plate.

The culture supernatant of the EBV-infected cells was added to the anti-hMPV antibody screening plate and reacted at room temperature for 1 hour, followed by washing with PBS-T three times. Next, anti-human IgG-Alexa 488 (Invitrogen Corp.) was diluted 1000-fold with PBS-T and 0.1% FCS, added thereto as a secondary antibody, and reacted at room temperature for 1 hour, followed by washing with PBS-T once. Next, 0.8 μg/ml DAPI and PBS-T were added thereto and reacted at room temperature for 10 minutes, followed by washing with PBS-T twice and the subsequent addition of 1×PBS(−).

The stained cells were photographed using In Cell Analyzer 2000 (GE Healthcare Japan Corp.). Each well in which the positive cells were detected was used as an antibody-positive well.

### 3. Confirmation of Antibody Isotype and Subclass

Each produced antibody was isotyped by the cell fluorescent immunostaining method using the culture supernatant of the separated antibody-producing cell clone. Specifically, the anti-hMPV antibody screening plate was used, and an antibody specific for each isotype and subclass was used as a secondary antibody. The isotypes and subclasses of the obtained 13 anti-hMPV antibodies are shown in Table 2 mentioned later.

### 4. Cloning of DNA Encoding Anti-hMPV Antibody

The total RNA of the antibody-producing cells was reverse-transcribed using oligo-dT primers. The obtained cDNA was used as a template in the PCR amplification of each antibody gene. The primers used in PCR were designed on the basis of the database of cDNAs encoding human IgG antibody H and L chains. In order to amplify the full-length H chain cDNA and L chain cDNA, the 5′ primer has a translation initiation point, and the 3′ primer has a translation termination point.

### 5. Determination of Amino Acid Sequence of Antibody Based on Nucleotide Sequence

The H chain and L chain cDNAs of each antibody amplified by PCR were inserted to plasmid vectors, and their nucleotide sequences were confirmed using an ABI sequencer. The signal sequence, H chain and L chain amino acid sequences, variable region amino acid sequences, and complementarity-determining region (CDR) amino acid sequences of the antibody were each determined from the obtained nucleotide sequences. In the CDR analysis, the method of Kabat (www.bioinf.org.uk: Dr. Andrew C. R. Martin's Group, Antibodies: General Information) was used. SEQ ID NOs of the amino acid sequences of the H and L chains of the obtained antibodies, the amino acid sequences of their variable regions, and the amino acid sequences of their complementarity-determining regions (CDRs) are each shown in Table 1.

### 6. Confirmation that Obtained Antibody Gene Encodes Anti-hMPV Antibody

The obtained H chain and L chain genes were respectively inserted to expression vectors. CHO-K1 cells were cotransfected with the expression vectors. The transfection was carried out using Lipofectamine LTX (Invitrogen Corp.) and Plus reagent (Invitrogen Corp.) under conditions recommended by the manufacturer. Two days later, the culture supernatant was recovered. Each antibody in the culture supernatant was confirmed to be human IgG by ELISA using a 96-well plate coated with an anti-human IgG antibody, and confirmed to bind to hMPV FP by the cell fluorescent immunostaining method using the anti-hMPV antibody screening plate.

### 7. Production of Antibody Protein

CHO-K1 cells were transfected with the obtained anti-hMPV antibody expression vector. The cells were cultured in the presence of a selection marker to obtain a CHO-K1 cell clone constitutively producing each antibody.

The CHO-K1 cells stably producing each antibody were cultured in a serum-free medium, and the culture supernatant was recovered. This culture supernatant was subjected to affinity purification using a protein A column to obtain a purified antibody. The column used was a prepack column of HiTrap rProtein A FF (GE Healthcare Japan Corp.). The purification was carried out under conditions recommended by the manufacturer. After the purification, the binding activity of each antibody against hMPV FP was confirmed using the anti-hMPV antibody screening plate. Also, an antibody H chain (approximately 50 kDa) and an antibody L chain (approximately 25 kDa) were confirmed by SDS-PAGE.

### 8. Neutralizing Activity Evaluation

Immunostaining Method:

The neutralizing activity was confirmed in the evaluation of each anti-hMPV antibody for its effectiveness. The neutralizing activity was evaluated on the basis of the rate of inhibition of hMPV infection of LLC-MK2 cells by the antibody. The hMPV strains used were JPS02-76 as a B1 strain, JPS05-21 as a B2 strain, JPS03-180 as an A1 strain, and JPS03-178 as an A2 strain.

The virus was prepared by the following method:

A virus solution was added to LLC-MK2 cells washed with 1×PBS(−) twice. After culture for 1 hour, EMEM containing 5 mM sucrose, 2 mM L-glutamine, and 0.5 μg/ml trypsin was added thereto as an infection medium, and the cells were cultured until CPE appeared. Then, uninfected LLC-ML2 cells and the infected cells were mixed at a ratio of 1:9 and cultured in an infection medium until CPE appeared to prepare seed-infected cells having 100% rate of infection. For a virus solution, uninfected LLC-MK2 cells and the seed-infected cells were mixed at a ratio of 1:9 and cultured in an infection medium until CPE appeared. The resulting culture supernatant was recovered. Then, the culture supernatant was centrifuged (20,000 g, 4° C., 2.5 hours), if necessary, to enrich the virus, which was then dispensed and cryopreserved at −80° C. until use.

The neutralizing activity was evaluated by the following method:

Six 4-fold dilution series of each purified anti-hMPV antibody were prepared from 10 μg/ml. The virus solution (0.8 to 1.4×10⁴ pfu/ml) and the purified antibody were mixed at a ratio of 1:1 and left at 25° C. for 1 hour. On the day before virus addition, LLC-MK2 cells were inoculated to a 96-well plate so as to reach 100% confluency. Before virus addition, the cells were washed with EMEM twice. The mixed solution of the virus solution and the purified antibody was added to LLC-MK2, and the cells were cultured for 1 hour in a CO₂ incubator. Then, the cells were washed with EMEM three times. EMEM containing 5 mM sucrose and 2 mM L-glutamine was added thereto, and the cells were cultured for 20 hours in a CO₂ incubator. In order to detect infected cells, the cells were fixed in 80% acetone, and the plate was blocked at room temperature for 1 hour by the addition of 1% BSA and 1×PBS(−). Next, a mouse anti-hMPV antibody (HMPV123, AbD Serotec) was diluted to 1 μg/ml with PBS-T and 0.1% goat serum, added thereto, and reacted at room temperature for 1 hour. After washing PBS-T three times, anti-mouse IgG-Alexa 488 (Invitrogen Corp.) diluted 3000-fold as a secondary antibody and DAPI diluted to 0.4 μg/ml with PBS-T and 0.1% goat serum were added thereto and reacted at room temperature for 1 hour. After washing with PBS-T three times, 1×PBS(−) was added thereto. The infected cells were photographed using In Cell Analyzer 2000 to count positive cells.

The negative control used in the neutralizing activity evaluation was a human monoclonal antibody (hIgG) having no specificity for hMPV. When the average number of infected cells in the presence of the negative control was defined as 0% rate of inhibition of infection, IC50 was calculated from the rate of inhibition of infection by each antibody at each concentration (calculated from antibody concentrations at two points spanning the 50% rate of inhibition, and the rates of inhibition) (Table 2).

Incidentally, a human-derived anti-hMPV antibody DSλ7 (Fab) has been shown to hardly exhibit neutralizing activity against B2 and A1 strains (neutralizing activity (IC60): >59 μg/mL (1180 nM) and 9.8 μg/mL (196 nM), respectively) (WO2008/043052 and J. Virol., 2007 (vol. 81) p. 8315). According to this report, human antibodies (DS1, DS6, ACN044, etc. (all, Fab)) exhibit no neutralizing activity against 3 strains (types A1, B1, and B2) except for the A2 strain (IC60 value: >8 μg/mL (160 nM)). In consideration of these factors, the antibody of the present invention having an IC50 value of approximately 1 μg/mL (approximately 6.7 nM) or lower against at least all of types A1, B1 and B2 is significantly superior to the human antibodies disclosed in WO2008/043052.

TABLE 2
Isotype of anti-hMPV antibody and IC50 against each hMPV strain

| | | IC50 (μg/ml) | | |
|---|---|---|---|---|
| | | hMPV-B1 | hMPV-B2 | hMPV-A1 |
| Ab ID. | isotype | JPS02-76 | JPS05-21 | JPS03-180 |
| EV046130 | IgG1/λ | 0.230 | 0.089 | 0.210 |
| EV046116 | IgG1/κ | 0.250 | 0.250 | 0.310 |
| EV046120 | IgG1/λ | 0.250 | 0.240 | 0.320 |
| EV046115b | IgG1/λ | 0.270 | 0.099 | 0.290 |
| EV046124 | IgG1/λ | 0.290 | 0.240 | 0.070 |
| EV046150 | IgG1/κ | 0.300 | 0.180 | 0.049 |
| EV046136 | IgG1/κ | 0.310 | 0.460 | 0.290 |
| EV046135 | IgG1/λ | 0.370 | 0.220 | 0.350 |
| EV046147 | IgG1/λ | 0.390 | 0.270 | 0.310 |
| EV046142 | IgG1/λ | 0.420 | 0.220 | 0.220 |
| EV046143 | IgG1/κ | 0.460 | 0.270 | 0.130 |
| EV046113 | IgG1/κ | 0.680 | 0.390 | 0.380 |
| EV046141 | IgG1/λ | 0.740 | 0.440 | 0.200 |

IC50, IC60, and IC90 further calculated from the data shown in Table 2 using data analysis software GraphPad Prism 6 are shown in Tables 3, 4, and 5, respectively. In these Tables 3, 4, and 5, neutralizing activity against JPS03-178 (type A2) was additionally shown, while positive controls used for neutralizing activity were prepared on the basis of information disclosed on a mouse antibody mAb338 against hMPV described in the patent literature JP2008-538353 (WO2006/110214) and a human antibody HMB3210 described in the patent literature WO2013/140247.

As a result of determining the IC50 (see Table 3) and IC60 (see Table 4) values by recalculation as mentioned above, all of the antibodies of the present invention also exhibited a neutralizing activity of approximately 1 μg/mL or lower (approximately 6.7 nM or lower) against types A1, B1, and B2. Thus, all of the antibodies of the present invention are significantly superior to the human antibodies disclosed in WO2008/043052. Their IC60 values against all types including type A2 were approximately 1 to 3 μg/mL or lower as a whole.

As is also evident from the results of Table 3, all of the antibodies of the present invention exhibited 50% inhibitory effect against all of the strains of hMPV type at approximately 2.3 μg/mL (approximately 15 nM) or lower. Thus, all of the antibodies of the present invention have high neutralizing activity. As seen from the results of Table 3, the anti-hMPV antibodies except for EV046115b, EV046130, EV046147, and EV046141 also exhibited 50% inhibitory effect (IC50) against all of the A1, A2, B1, and B2 strains at approximately 1 μg/mL (approximately 6.7 nM) or lower. Furthermore, all of EV046120, EV046124, EV046135, EV046142, EV046143, and EV046150 exhibited 50% inhibitory effect against hMPV type A2 at approximately 0.1 μg/mL (approximately 0.67 nM) or lower. Particularly, EV046135, EV046143, and EV046150 exhibited 50% inhibitory effect at approximately 0.03 μg/mL (approximately 0.2 nM) or lower. These antibodies were significantly superior even to HMB3210 (139.3 ng/mL).

As also seen from the results about the IC90 values in Table 5, all of the antibodies except for EV046115b exhibited 90% inhibitory effect (IC90) against all of types A1, A2, B1, and B2 at approximately 3.5 μg/mL (approximately 23 nM) or lower. Among them, particularly, EV046124 and EV046150 exhibited 90% inhibitory effect (IC90) against all of types A1, A2, B1, and B2 at approximately 1 μg/mL (approximately 6.7 nM) or lower. Thus, these antibodies have much higher neutralizing activity even than that of HMB3210 (approximately 3.6 μg/mL or lower). Furthermore, all of EV046124, EV046143, and EV046150 exhibited a neutralizing activity of 188.1 ng/mL, 96.71 ng/mL, and 101.1 ng/mL, respectively, against hMPV type A2 and thus had a neutralizing activity as very high as approximately 0.2 μg/mL (approximately 1.33 nM) or lower. Particularly, both EV046143 and EV046150 had an IC90 value of approximately 0.1 μg/mL (approximately 0.67 nM). These antibodies were significantly superior even to HMB3210 (approximately 0.58 μg/mL).

TABLE 3
IC50 of anti-hMPV antibody against each hMPV strain

| | IC50 (ng/ml) | | | |
|---|---|---|---|---|
| | MPV-A1 | MPV-A2 | MPV-B1 | MPV-B2 |
| Ab ID. | JPS03-180 | JPS03-178 | JPS02-76 | JPS05-21 |
| EV046113 | 418.0 | 645.1 | 716.2 | 490.7 |
| EV046115b | 322.4 | 2284 | 249.7 | 107.6 |
| EV046116 | 351.1 | 189.8 | 330.9 | 279.0 |
| EV046120 | 300.1 | 97.13 | 241.3 | 249.4 |
| EV046124 | 76.73 | 40.67 | 352.8 | 254.9 |
| EV046130 | 219.5 | 1950 | 302.3 | 104.8 |
| EV046135 | 348.2 | 31.56 | 463.9 | 212.6 |
| EV046136 | 318.1 | 318.1 | 352.9 | 537.0 |
| EV046141 | 213.5 | 2113 | 718.7 | 524.0 |
| EV046142 | 211.8 | 61.35 | 523.3 | 272.6 |
| EV046143 | 156.6 | 19.81 | 605.7 | 272.4 |
| EV046147 | 326.1 | 1975 | 425.4 | 252.7 |
| EV046150 | 61.81 | 29.78 | 314.3 | 202.1 |
| mAb338 | 124.7 | 180.3 | 281.1 | 221.0 |
| HMB3210 | 388.8 | 139.3 | 138.8 | 40.81 |

TABLE 4
IC60 of anti-hMPV antibody against each hMPV strain

| | IC60 (ng/ml) | | | |
|---|---|---|---|---|
| | MPV-A1 | MPV-A2 | MPV-B1 | MPV-B2 |
| Ab ID. | JPS03-180 | JPS03-178 | JPS02-76 | JPS05-21 |
| EV046113 | 521.8 | 759.6 | 818.8 | 652.1 |
| EV046115b | 399.5 | 2709 | 376.0 | 133.4 |
| EV046116 | 427.9 | 247.4 | 497.4 | 341.3 |
| EV046120 | 421.0 | 138.0 | 344.4 | 301.3 |
| EV046124 | 102.3 | 53.93 | 423.3 | 305.0 |
| EV046130 | 254.0 | 2010 | 354.0 | 131.6 |
| EV046135 | 440.0 | 47.12 | 571.1 | 268.4 |
| EV046136 | 358.5 | 358.5 | 419.3 | 669.8 |
| EV046141 | 276.1 | 2184 | 882.9 | 570.8 |
| EV046142 | 242.7 | 97.11 | 641.0 | 365.7 |
| EV046143 | 186.4 | 26.55 | 627.3 | 363.1 |
| EV046147 | 405.9 | 2060 | 513.8 | 297.7 |
| EV046150 | 82.31 | 37.32 | 360.5 | 229.4 |
| mAb338 | 160.0 | 239.0 | 338.0 | 261.1 |
| HMB3210 | 585.6 | 181.2 | 190.1 | 48.13 |

TABLE 5
IC90 of anti-hMPV antibody against each hMPV strain

| | IC90 (ng/ml) | | | |
|---|---|---|---|---|
| | MPV-A1 | MPV-A2 | MPV-B1 | MPV-62 |
| Ab ID. | JPS03-180 | JPS03-178 | JPS02-76 | JPS05-21 |
| EV046113 | 1393 | 1558 | 1480 | 2289 |
| EV046115b | 1034 | 5745 | 2304 | 344.7 |
| EV046116 | 1030 | 800.4 | 3005 | 828.3 |
| EV046120 | 1883 | 652.6 | 1651 | 693.5 |
| EV046124 | 364.6 | 188.1 | 952.3 | 676.8 |
| EV046130 | 485.8 | 2296 | 714.4 | 358.2 |
| EV046135 | 1235 | 277.2 | 1437 | 748.0 |
| EV046136 | 605.3 | 605.3 | 895.0 | 1778 |
| EV046141 | 863.0 | 2523 | 2182 | 835.9 |
| EV046142 | 443.1 | 737.6 | 1567 | 1346 |
| EV046143 | 401.6 | 96.71 | 734.5 | 1295 |
| EV046147 | 1072 | 2407 | 1182 | 612.1 |
| EV046150 | 292.0 | 101.1 | 662.8 | 403.6 |
| mAb338 | 498.4 | 856.3 | 772.0 | 547.0 |
| HMB3210 | 3569 | 578.7 | 847.7 | 99.53 |

### 9. Evaluation of Cell-to-Cell Spread Inhibitory Activity

The cell-to-cell spread inhibitory activity was further evaluated as the evaluation of each anti-hMPV antibody for its effectiveness.

hMPV infection requires the cleavage of FP. LLC-MK2 cells or VERO cells, which serve as host cells for in vitro hMPV infection, lack the activity of cleaving FP. For this reason, trypsin is generally added to a medium. However, antibody deactivation by trypsin is of concern to a system in which antibodies are continuously added in order to evaluate cell-to-cell spread inhibitory activity. In this respect, membrane-type serine protease (TMPRSS2) reportedly having the activity of cleaving FP was introduced into LLC-MK2 cells to prepare host cells stably expressing TMPRSS2 (Shirogane et al. 2008 J. Virol. 82: 8942-8946).

The obtained cells stably expressing TMPRSS2 were infected by hMPV. As a result of spreading the hMPV infection under trypsin-free conditions, the number of infected cells was increased, and foci were confirmed.

The actual evaluation of the cell-to-cell spread inhibitory activity was conducted as follows: the cells stably expressing TMPRSS2 were infected by hMPV. After a lapse of 4 hours, six 4-fold dilution series of each antibody starting at 40 μg/ml were added to the cells. After 72 hours, the cells were fixed in the same way as in the neutralizing activity evaluation, and the infected cells were detected.

The negative control used in the cell-to-cell spread inhibitory activity evaluation was a human monoclonal antibody (hIgG) having no specificity for hMPV. The positive control used in this evaluation was a synthesized antibody mAb338 against hMPV as described in the patent literature JP2008-538353. When the average number of infected cells in the presence of the negative control at respective concentrations was defined as 0% rate of inhibition of cell-to-cell spread and the number of infected cells in the presence of the positive control at 40 μg/ml was defined as 100% rate of inhibition of cell-to-cell spread, the rate of inhibition of infection by each antibody at each concentration was calculated. Its IC50 and IC90 (ng/ml) were calculated using data analysis software GraphPad Prism 6. The results are shown in Table 6.

TABLE 6
Cell-to-cell spread inhibitory activity of anti-hMPV antibody

| | MPV-B1 (JPS02-76) | |
|---|---|---|
| Ab ID. | IC50 (ng/ml) | IC90 (ng/ml) |
| EV046113 | 107.6 | 707.4 |
| EV046115b | 134.5 | 617.0 |
| EV046116 | 54.00 | 132.2 |
| EV046120 | 13.16 | 66.88 |
| EV046124 | 48.18 | 259.5 |
| EV046130 | 34.86 | 147.3 |
| EV046135 | 30.77 | 230.5 |
| EV046136 | 52.22 | 211.9 |
| EV046141 | 36.27 | 171.0 |
| EV046142 | 12.34 | 66.41 |
| EV046143 | 57.19 | 885.7 |
| EV046147 | 113.9 | 506.1 |
| EV046150 | 16.64 | 64.95 |
| mAb338 | 21.54 | 93.75 |

### 10. Affinity Analysis

The affinity analysis was conducted as the evaluation of each anti-hMPV antibody for its binding activity.

The antigens used in the analysis were prepared as follows: a gene encoding FP-TM(−)-His, which was His-Tag-added hMPV (JPS02-76) FP lacking the transmembrane region (amino acid residues 491 to 539 of FP), was cloned into an expression vector. CHO-K1 cells were transfected with the FP-TM(−)-His expression vector. The cells were cultured in the presence of a selection marker to obtain a CHO-K1 cell clone stably expressing FP-TM(−)-His. The culture supernatant of the cells stably expressing FP-TM(−)-His was recovered. FP-TM(−)-His was purified using Ni-Sepharose 6 Fast Flow carrier (GE Healthcare Japan Corp.). After the purification, imidazole was removed from FP-TM(−)-His by gel filtration, and the resulting protein was quantified and then used in affinity analysis.

The affinity analysis was conducted using Biacore T-200. Anti-human IgG was immobilized onto a sensor chip using human IgG capture kit (GE Healthcare Japan Corp.). Each anti-hMPV antibody was captured thereon and used as a ligand. The analyte used was the FP-TM(−)-His thus purified. The affinity of each anti-hMPV antibody for hMPV (JPS02-76) FP is shown in Table 7. All of the anti-hMPV antibodies exhibited binding activity as very high as a K_{D} value of 10 nM or lower against FP (Table 7). The affinity of EV046115b and EV046147 was immeasurable (ND) due to strong aggregation reaction on the sensor chip.

TABLE 7
Results of affinity analysis of anti-hMPV antibody

| | Affiniy (MPV-B1:JPS02-76) | | |
|---|---|---|---|
| Ab ID. | Ka (1/Ms) | Kd (1/s) | KD (M) |
| EV046113 | 4.632E+5 | 1.227E−4 | 2.648E−10 |
| Ev046115b | N.D. | N.D. | N.D. |
| EV046116 | 2.521E+5 | 4.077E−4 | 1.618E−9 |
| EV046120 | 2.898E+5 | 6.169E−4 | 2.115E−9 |
| EV046124 | 3.768E+5 | 2.255E−4 | 5.993E−10 |
| EV046130 | 5.873E+5 | 1.532E−3 | 2.642E−9 |
| EV046135 | 4.139E+5 | 3.649E−4 | 9.291E−10 |
| EV046136 | 3.914E+5 | 3.651E−4 | 9.501E−10 |
| EV046141 | 7.156E+5 | 1.179E−3 | 1.810E−9 |
| EV046142 | 2.755E+5 | 1.085E−4 | 4.249E−10 |
| EV046143 | 5.809E+5 | 9.379E−5 | 1.640E−10 |
| EV046147 | N.D. | N.D. | N.D. |
| EV046150 | 3.914E+5 | 9.635E−5 | 2.484E−10 |
| mAb338 | 4.344E+5 | 2.283E−4 | 5.315E−10 |

(N.D.: immeasurable due to antibody aggregation reaction on the sensor chip)

### 11. Competition Assay

The competition assay of each anti-hMPV antibody was conducted.

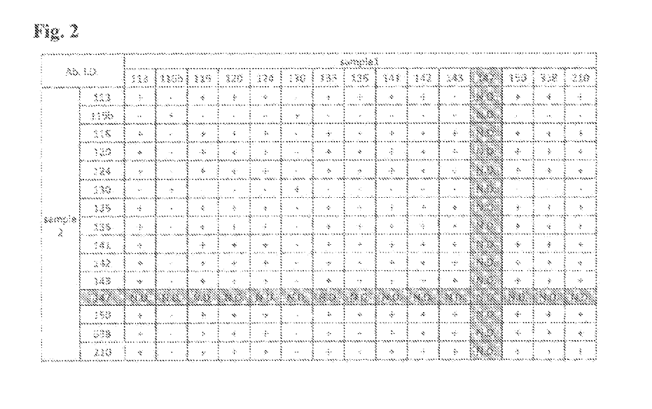

The antigen used in in the analysis was the culture supernatant of the cells stably expressing FP-TM(−)-His as shown in the affinity analysis. The assay was conducted using Biacore T-200. After capturing of FP-TM(−)-His onto Sensor Chip NTA (GE Healthcare Japan Corp.), an anti-hMPV antibody was bound as Sample 1 to the sensor chip until a saturation level. Next, another anti-MPV antibody was bound thereto as Sample 2. The presence or absence of competition was determined on the basis of whether or not the binding of the anti-hMPV antibody as Sample 2 was inhibited. The competition assay results are shown in FIG. 2. The binding of EV046147 was immeasurable due to strong aggregation reaction on the sensor chip.

### 12. Epitope Mapping

For the epitope grouping of the anti-hMPV antibodies, deletion mutant binding assay was conducted.

The prepared deletion mutants are shown in FIG. 3. HA-V5-Full is hMPV (JPS02-76) FP containing HA-Tag (HA; TMYPYDVPDYA) added downstream of the signal peptide (SP) and a linker moiety (SLEGPRFE) and V5-Tag (V5; GKPIPNPLLGLDST) added downstream of the C-tail (C). The full-length nucleotide sequence of the HA-V5-Full used and an amino acid sequence encoded thereby are shown in SEQ ID NOs: 133 and 134, respectively.

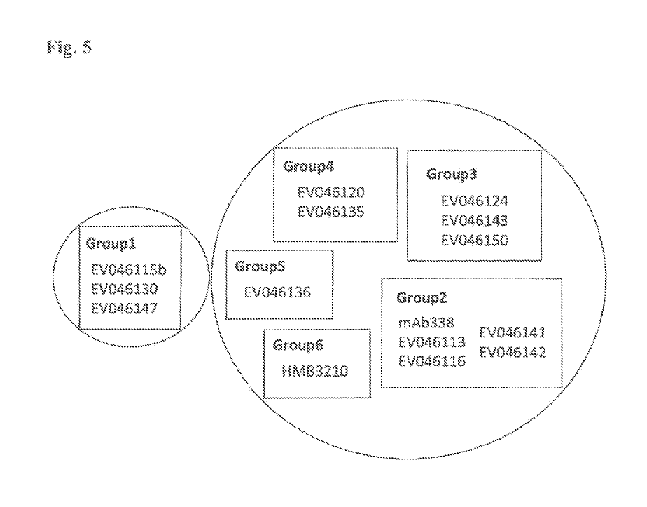

The gene of each deletion mutant was cloned into an expression vector. The binding of each anti-hMPV antibody was detected in the same way as in the cell fluorescent immunostaining screening. Also, an anti-HA antibody and an anti-V5 antibody were used to confirm the expression of the deletion mutants themselves. The deletion mutant binding assay results are shown in FIG. 4. The epitope grouping results together with the competition assay results are shown in FIG. 5.

For the competition assay and the epitope mapping, a mouse anti-hMPV antibody mAb338 (J. Virol., 2008 (vol. 89) p. 3113) and a human antibody HMB3210 were used as controls.

These results demonstrated that: HMB3210 does not belong to any of the epitope groups of the antibody of the present invention; and mAb338 belongs to the epitope group 2 of the antibody of the present invention.

### INDUSTRIAL APPLICABILITY

The anti-human metapneumovirus (hMPV) antibody of the present invention is useful in uses such as a pharmaceutical composition for the prevention or treatment of a disease involving the hMPV.

1. An antibody capable of specifically binding to human metapneumovirus F protein and neutralizing its biological activity, or an antigen-binding fragment thereof, comprising a heavy chain variable region and a light chain variable region wherein
(i) the heavy chain variable region comprises:
(a) the amino acid sequence of heavy chain CDR1 selected from the group consisting of the amino acid sequences of SEQ ID NOs: 3, 13, 23, 33, 43, 53, 63, 73, 83, 93,
103, 113, and 123 and amino acid sequences derived from any of these amino acid sequences by the deletion, substitution, insertion, and/or addition mutations of one to several amino acid residues,
(b) the amino acid sequence of heavy chain CDR2 selected from the group consisting of the amino acid sequences of SEQ ID NOs: 4, 14, 24, 34, 44, 54, 64, 74, 84, 94, 104, 114, and 124 and amino acid sequences derived from any of these amino acid sequences by the deletion, substitution, insertion, and/or addition mutations of one to several amino acid residues, and
(c) the amino acid sequence of heavy chain CDR3 selected from the group consisting of the amino acid sequences of SEQ ID NOs: 5, 15, 25, 35, 45, 55, 65, 75, 85, 95, 105, 115, and 125 and amino acid sequences derived from any of these amino acid sequences by the deletion, substitution, insertion, and/or addition mutations of one to several amino acid residues, and
(ii) the light chain variable region comprises:
(a) the amino acid sequence of light chain CDR1 selected from the group consisting of the amino acid sequences of SEQ ID NOs: 8, 18, 28, 38, 48, 58, 68, 78, 88, 98, 108, 118, and 128 and amino acid sequences derived from any of these amino acid sequences by the deletion, substitution, insertion, and/or addition mutations of one to several amino acid residues,
(b) the amino acid sequence of light chain CDR2 selected from the group consisting of the amino acid sequences of SEQ ID NOs: 9, 19, 29, 39, 49, 59, 69, 79, 89, 99, 109, 119, and 129 and amino acid sequences derived from any of these amino acid sequences by the deletion, substitution, insertion, and/or addition mutations of one to several amino acid residues, and
(c) the amino acid sequence of light chain CDR3 selected from the group consisting of the amino acid sequences of SEQ ID NOs: 10, 20, 30, 40, 50, 60, 70, 80, 90, 100, 110, 120, and 130 and amino acid sequences derived from any of these amino acid sequences by the deletion, substitution, insertion, and/or addition mutations of one to several amino acid residues.

2. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 3,
(b) the amino acid sequence of heavy chain CDR2 comprising the amino acid sequence of SEQ ID NO: 4, and
(c) the amino acid sequence of heavy chain CDR3 comprising the amino acid sequence of SEQ ID NO: 5, and
(ii) the light chain variable region comprises:
(a) the amino acid sequence of light chain CDR1 comprising the amino acid sequence of SEQ ID NO: 8,
(a) the amino acid sequence of light chain CDR1 comprising the amino acid sequence of SEQ ID NO: 8,
(b) the amino acid sequence of light chain CDR2 comprising the amino acid sequence of SEQ ID NO: 9, and
(c) the amino acid sequence of light chain CDR3 comprising the amino acid sequence of SEQ ID NO: 10.

3. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 13,
(b) the amino acid sequence of heavy chain CDR2 comprising the amino acid 30 sequence of SEQ ID NO: 14, and
(c) the amino acid sequence of heavy chain CDR3 comprising the amino acid sequence of SEQ ID NO: 15, and
(ii) the light chain variable region comprises:
(a) the amino acid sequence of light chain CDR1 comprising the amino acid sequence of SEQ ID NO: 18,
(b) the amino acid sequence of light chain CDR2 comprising the amino acid sequence of SEQ ID NO: 19, and
(c) the amino acid sequence of light chain CDR3 comprising the amino acid sequence of SEQ ID NO: 20.

4. The antibody or an antigen-binding fragment thereof according to claim 1,
wherein
(i) the heavy chain variable region comprises:
(a) the amino acid sequence of heavy chain CDR1 comprising the amino acid sequence of SEQ ID NO: 23,
(b) the amino acid sequence of heavy chain CDR2 comprising the amino acid sequence of SEQ ID NO: 24, and
(c) the amino acid sequence of heavy chain CDR3 comprising the amino acid sequence of SEQ ID NO: 25, and
(ii) the light chain variable region comprises:
(a) the amino acid sequence of light chain CDR1 comprising the amino acid sequence of SEQ ID NO: 28,
(b) the amino acid sequence of light chain CDR2 comprising the amino acid sequence of SEQ ID NO: 29, and
(c) the amino acid sequence of light chain CDR3 comprising the amino acid sequence of SEQ ID NO: 30.

5. The antibody or an antigen-binding fragment thereof according to claim 1,
wherein
(i) the heavy chain variable region comprises:
(a) the amino acid sequence of heavy chain CDR1 comprising the amino acid sequence of SEQ ID NO: 33,
(b) the amino acid sequence of heavy chain CDR2 comprising the amino acid sequence of SEQ ID NO: 34, and
(c) the amino acid sequence of heavy chain CDR3 comprising the amino acid sequence of SEQ ID NO: 35, and
(ii) the light chain variable region comprises:
(a) the amino acid sequence of light chain CDR1 comprising the amino acid sequence of SEQ ID NO: 38, (b) the amino acid sequence of light chain CDR2 comprising the amino acid
sequence of SEQ ID NO: 39, and
(c) the amino acid sequence of light chain CDR3 comprising the amino acid sequence of SEQ ID NO: 40.

6. The antibody or an antigen-binding fragment thereof according to claim 1,
wherein
(i) the heavy chain variable region comprises:
(a) the amino acid sequence of heavy chain CDR1 comprising the amino acid sequence of SEQ ID NO: 43,
(b) the amino acid sequence of heavy chain CDR2 comprising the amino acid sequence of SEQ ID NO: 44, and
(c) the amino acid sequence of heavy chain CDR3 comprising the amino acid sequence of SEQ ID NO: 45, and
(ii) the light chain variable region comprises:
(a) the amino acid sequence of light chain CDR1 comprising the amino acid sequence of SEQ ID NO: 48,
(b) the amino acid sequence of light chain CDR2 comprising the amino acid sequence of SEQ ID NO: 49, and
(c) the amino acid sequence of light chain CDR3 comprising the amino acid sequence of SEQ ID NO: 50.

7. The antibody or an antigen-binding fragment thereof according to claim 1,
wherein
(i) the heavy chain variable region comprises
(a) the amino acid sequence of heavy chain CDR1 comprising the amino acid sequence of SEQ ID NO: 53,
(b) the amino acid sequence of heavy chain CDR2 comprising the amino acid sequence of SEQ ID NO: 54, and
(c) the amino acid sequence of heavy chain CDR3 comprising the amino acid sequence of SEQ ID NO: 55, and
(ii) the light chain variable region comprises:
(a) the amino acid sequence of light chain CDR1 comprising the amino acid sequence of SEQ ID NO: 58,
(b) the amino acid sequence of light chain CDR2 comprising the amino acid sequence of SEQ ID NO: 59, and
(c) the amino acid sequence of light chain CDR3 comprising the amino acid sequence of SEQ ID NO: 60.

8. The antibody or an antigen-binding fragment thereof according to claim 1,
wherein
(i) the heavy chain variable region comprises:
(a) the amino acid sequence of heavy chain CDR1 comprising the amino acid sequence of SEQ ID NO: 63,
(b) the amino acid sequence of heavy chain CDR2 comprising the amino acid sequence of SEQ ID NO: 64, and
(c) the amino acid sequence of heavy chain CDR3 comprising the amino acid sequence of SEQ ID NO: 65, and
(ii) the light chain variable region comprises:
(a) the amino acid sequence of light chain CDR1 comprising the amino acid sequence of SEQ ID NO: 68,
(b) the amino acid sequence of light chain CDR2 comprising the amino acid sequence of SEQ ID NO: 69, and
(c) the amino acid sequence of light chain CDR3 comprising the amino acid sequence of SEQ ID NO: 70.

9. The antibody or an antigen-binding fragment thereof according to claim 1,
wherein
(i) the heavy chain variable region comprises:
(a) the amino acid sequence of heavy chain CDR1 comprising the amino acid sequence of SEQ ID NO: 73,
(b) the amino acid sequence of heavy chain CDR2 comprising the amino acid sequence of SEQ ID NO: 74, and
(c) the amino acid sequence of heavy chain CDR3 comprising the amino acid sequence of SEQ ID NO: 75, and
(ii) the light chain variable region comprises:
(a) the amino acid sequence of light chain CDR1 comprising the amino acid sequence of SEQ ID NO: 78, (b) the amino acid sequence of light chain CDR2 comprising the amino acid sequence of SEQ ID NO: 79, and
(c) the amino acid sequence of light chain CDR3 comprising the amino acid sequence of SEQ ID NO: 80.

10. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 83,
(b) the amino acid sequence of heavy chain CDR2 comprising the amino acid sequence of SEQ ID NO: 84, and
(c) the amino acid sequence of heavy chain CDR3 comprising the amino acid sequence of SEQ ID NO: 85, and
(ii) the light chain variable region comprises:
(a) the amino acid sequence of light chain CDR1 comprising the amino acid sequence of SEQ ID NO: 88,
(b) the amino acid sequence of light chain CDR2 comprising the amino acid sequence of SEQ ID NO: 89, and
(c) the amino acid sequence of light chain CDR3 comprising the amino acid sequence of SEQ ID NO: 90.

11. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 93,
(b) the amino acid sequence of heavy chain CDR2 comprising the amino acid sequence of SEQ ID NO: 94, and
(c) the amino acid sequence of heavy chain CDR3 comprising the amino acid sequence of SEQ ID NO: 95, and
(ii) the light chain variable region comprises:
(a) the amino acid sequence of light chain CDR1 comprising the amino acid sequence of SEQ ID NO: 98, (b) the amino acid sequence of light chain CDR2 comprising the amino acid sequence of SEQ ID NO: 99, and
(c) the amino acid sequence of light chain CDR3 comprising the amino acid sequence of SEQ ID NO: 100.

12. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 103,
(b) the amino acid sequence of heavy chain CDR2 comprising the amino acid sequence of SEQ ID NO: 104, and
(c) the amino acid sequence of heavy chain CDR3 comprising the amino acid sequence of SEQ ID NO: 105, and
(ii) the light chain variable region comprises:
(a) the amino acid sequence of light chain CDR1 comprising the amino acid sequence of SEQ ID NO: 108,
(b) the amino acid sequence of light chain CDR2 comprising the amino acid sequence of SEQ ID NO: 109, and
(c) the amino acid sequence of light chain CDR3 comprising the amino acid sequence of SEQ ID NO: 110.

13. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 113,
(b) the amino acid sequence of heavy chain CDR2 comprising the amino acid sequence of SEQ ID NO: 114, and
(c) the amino acid sequence of heavy chain CDR3 comprising the amino acid sequence of SEQ ID NO: 115, and
(ii) the light chain variable region comprises:
(a) the amino acid sequence of light chain CDR1 comprising the amino acid sequence of SEQ ID NO: 118, (b) the amino acid sequence of light chain CDR2 comprising the amino acid sequence of SEQ ID NO: 119, and
(c) the amino acid sequence of light chain CDR3 comprising the amino acid sequence of SEQ ID NO: 120.

14. The antibody or an antigen-binding fragment thereof according to claim 1, wherein
(i) the heavy chain variable region comprises:
(a) the amino acid sequence of heavy chain CDR1 comprising the amino acid sequence of SEQ ID NO: 123,
(b) the amino acid sequence of heavy chain CDR2 comprising the amino acid sequence of SEQ ID NO: 124, and
(c) the amino acid sequence of heavy chain CDR3 comprising the amino acid sequence of SEQ ID NO: 125, and
(ii) the light chain variable region comprises:
(a) the amino acid sequence of light chain CDR1 comprising the amino acid sequence of SEQ ID NO: 128,
(b) the amino acid sequence of light chain CDR2 comprising the amino acid sequence of SEQ ID NO: 129, and
(c) the amino acid sequence of light chain CDR3 comprising the amino acid sequence of SEQ ID NO: 130.

15. The antibody or an antigen-binding fragment thereof according to claim 1, wherein the antibody comprises:
(a) a heavy chain variable region (VH) selected from the group consisting of the amino acid sequences of SEQ ID NOs: 2, 12, 22, 32, 42, 52, 62, 72, 82, 92, 102, 112, and 122 and amino acid sequences having 80% or higher identity to any of these amino acid sequences, and
(b) a light chain variable region (VL) selected from the group consisting of the amino 30 acid sequences of SEQ ID NOs: 7, 17, 27, 37, 47, 57, 67, 77, 87, 97, 107, 117, and 127 and amino acid sequences having 80% or higher identity to any of these amino acid sequences.

16. The antibody or an antigen-binding fragment thereof according to claim 15, wherein the antibody comprises:
(a) a heavy chain variable region (VH) comprising the amino acid sequence of SEQ ID NO: 2 or an amino acid sequence having 80% or higher identity to the amino acid sequence of SEQ ID NO: 2, and
(b) a light chain variable region (VL) comprising the amino acid sequence of SEQ ID NO: 7 or an amino acid sequence having 80% or higher identity to the amino acid sequence of SEQ ID NO: 7.

17. The antibody or an antigen-binding fragment thereof according to claim 15, wherein the antibody comprises:
(a) a heavy chain variable region (VH) comprising the amino acid sequence of SEQ ID NO: 12 or an amino acid sequence having 80% or higher identity to the amino acid sequence of SEQ ID NO: 12, and
(b) a light chain variable region (VL) comprising the amino acid sequence of SEQ ID NO: 17 or an amino acid sequence having 80% or higher identity to the amino acid sequence of SEQ ID NO: 17.

18. The antibody or an antigen-binding fragment thereof according to claim 15, wherein the antibody comprises:
(a) a heavy chain variable region (VH) comprising the amino acid sequence of SEQ ID NO: 22 or an amino acid sequence having 80% or higher identity to the amino acid sequence of SEQ ID NO: 22, and
(b) a light chain variable region (VL) comprising the amino acid sequence of SEQ ID NO: 27 or an amino acid sequence having 80% or higher identity to the amino acid sequence of SEQ ID NO: 27.

19. The antibody or an antigen-binding fragment thereof according to claim 15, wherein the antibody comprises:
(a) a heavy chain variable region (VH) comprising the amino acid sequence of SEQ ID NO: 32 or an amino acid sequence having 80% or higher identity to the amino acid sequence of SEQ ID NO: 32, and
(b) a light chain variable region (VL) comprising the amino acid sequence of SEQ ID NO: 37 or an amino acid sequence having 80% or higher identity to the amino acid sequence of SEQ ID NO: 37.

20. The antibody or an antigen-binding fragment thereof according to claim 15, wherein the antibody comprises:
(a) a heavy chain variable region (VH) comprising the amino acid sequence of SEQ ID NO: 42 or an amino acid sequence having 80% or higher identity to the amino acid sequence of SEQ ID NO: 42, and
(b) a light chain variable region (VL) comprising the amino acid sequence of SEQ ID NO: 47 or an amino acid sequence having 80% or higher identity to the amino acid sequence of SEQ ID NO: 47.

21. The antibody or an antigen-binding fragment thereof according to claim 15, wherein the antibody comprises:
(a) a heavy chain variable region (VH) comprising the amino acid sequence of SEQ ID NO: 52 or an amino acid sequence having 80% or higher identity to the amino acid sequence of SEQ ID NO: 52, and
(b) a light chain variable region (VL) comprising the amino acid sequence of SEQ ID NO: 57 or an amino acid sequence having 80% or higher identity to the amino acid sequence of SEQ ID NO: 57.

22. The antibody or an antigen-binding fragment thereof according to claim 15, wherein the antibody comprises:
(a) a heavy chain variable region (VH) comprising the amino acid sequence of SEQ ID NO: 62 or an amino acid sequence having 80% or higher identity to the amino acid sequence of SEQ ID NO: 62, and
(b) a light chain variable region (VL) comprising the amino acid sequence of SEQ ID 25 NO: 67 or an amino acid sequence having 80% or higher identity to the amino acid sequence of SEQ ID NO: 67.

23. The antibody or an antigen-binding fragment thereof according to claim 15, wherein the antibody comprises:
(a) a heavy chain variable region (VH) comprising the amino acid sequence of SEQ ID NO: 72 or an amino acid sequence having 80% or higher identity to the amino acid sequence of SEQ ID NO: 72, and
(b) a light chain variable region (VL) comprising the amino acid sequence of SEQ ID NO: 77 or an amino acid sequence having 80% or higher identity to the amino acid 35 sequence of SEQ ID NO: 77.

24. The antibody or an antigen-binding fragment thereof according to claim 15, wherein the antibody comprises:
(a) a heavy chain variable region (VH) comprising the amino acid sequence of SEQ ID NO: 82 or an amino acid sequence having 80% or higher identity to the amino acid sequence of SEQ ID NO: 82, and
(b) a light chain variable region (VL) comprising the amino acid sequence of SEQ ID NO: 87 or an amino acid sequence having 80% or higher identity to the amino acid sequence of SEQ ID NO: 87.

25. The antibody or an antigen-binding fragment thereof according to claim 15, wherein the antibody comprises:
(a) a heavy chain variable region (VH) comprising the amino acid sequence of SEQ ID NO: 92 or an amino acid sequence having 80% or higher identity to the amino acid sequence of SEQ ID NO: 92, and
(b) a light chain variable region (VL) comprising the amino acid sequence of SEQ ID NO: 97 or an amino acid sequence having 80% or higher identity to the amino acid sequence of SEQ ID NO: 97.

26. The antibody or an antigen-binding fragment thereof according to claim 15, wherein the antibody comprises:
(a) a heavy chain variable region (VH) comprising the amino acid sequence of SEQ ID NO: 102 or an amino acid sequence having 80% or higher identity to the amino acid sequence of SEQ ID NO: 102, and
(b) a light chain variable region (VL) comprising the amino acid sequence of SEQ ID NO: 107 or an amino acid sequence having 80% or higher identity to the amino acid sequence of SEQ ID NO: 107.

27. The antibody or an antigen-binding fragment thereof according to claim 15, wherein the antibody comprises:
(a) a heavy chain variable region (VH) comprising the amino acid sequence of SEQ ID NO: 112 or an amino acid sequence having 80% or higher identity to the amino acid sequence of SEQ ID NO: 112, and
(b) a light chain variable region (VL) comprising the amino acid sequence of SEQ ID NO: 117 or an amino acid sequence having 80% or higher identity to the amino acid sequence of SEQ ID NO: 117.

28. The antibody or an antigen-binding fragment thereof according to claim 15, wherein the antibody comprises:
(a) a heavy chain variable region (VH) comprising the amino acid sequence of SEQ ID NO: 122 or an amino acid sequence having 80% or higher identity to the amino acid sequence of SEQ ID NO: 122, and
(b) a light chain variable region (VL) comprising the amino acid sequence of SEQ ID NO: 127 or an amino acid sequence having 80% or higher identity to the amino acid sequence of SEQ ID NO: 127.

29. The antibody or an antigen-binding fragment thereof according to claim 1, wherein an epitope of the antibody belongs to any of epitope groups Group 1 typified by anti-hMPV antibodies EV046115b, EV046130, and EV046147, Group 2 typified by anti-hMPV antibodies EV046113, EV046116, EV046141, and EV0461142, Group 3 typified by anti-hMPV antibodies EV046124, EV046143, and EV046150, Group 4 typified by anti-hMPV antibodies EV046120 and EV046135, and Group 5 typified by an anti-hMPV antibody EV046136 in epitope grouping according to binding activity against a partially deleted peptide of the F protein.

30. The antibody or an antigen-binding fragment thereof according to claim 1, wherein said antibody comprises an epitope, and wherein the epitope of the antibody belongs to any of epitope groups Group 2 typified by anti-hMPV antibodies EV046113, EV046116, EV046141, and EV0461142, Group 3 typified by anti-hMPV antibodies EV046124, EV046143, and EV046150, Group 4 typified by anti-hMPV antibodies EV046120 and EV046135, and Group 5 typified by an anti-hMPV antibody EV046136 in epitope grouping according to binding activity against a partially deleted peptide of the F protein.

31. The antibody or an antigen-binding fragment thereof according to claim 1, wherein said antibody comprises an epitope, and wherein the epitope of the antibody belongs to an epitope group Group 3 typified by anti-hMPV antibodies EV046124, EV046143, and EV046150 in epitope grouping according to binding activity against a partially deleted peptide of the F protein.

32. The antibody or an antigen-binding fragment thereof according to claim 1, wherein the antibody is of IgG1 (x) or IgG1 (A.) class (subclass).

33. The antibody or an antigen-binding fragment thereof according to claim 1, wherein the antibody has a neutralizing activity (IC50) of 1 μg/mL (approximately 6.7 nM) or lower against any of human metapneumovirus strains JPS02-76 (type B1), JPS05-21 (type B2), and JPS03-180 (type A1).

34. The antibody or an antigen-binding fragment thereof according to claim 1, wherein the antibody has a neutralizing activity (IC50) of approximately 1 μg/mL (approximately 6.7 nM) or lower against any of human metapneumovirus strains JPS03-180 (type A1), JPS03-1 78 (type A2), JPS02-76 (type B1), and JPS05-21 (type B2).

35. The antibody or an antigen-binding fragment thereof according to claim 1, wherein the antibody has a neutralizing activity (IC90) of approximately 2 μg/mL (approximately 13.3 nM) or lower against any of human metapneumovirus strains JPS03-1 80 (type A1), JPS03-1 78 (type A2), JPS02-76 (type B1), and JPS05-21 (type B2).

36. The antibody or an antigen-binding fragment thereof according to claim 1, wherein the antibody has a neutralizing activity (IC50) of approximately 0.1 μg/mL (approximately 0.67 nM) or lower against a human metapneumovirus JPS03-1 78 strain (type A2).

37. A pharmaceutical composition comprising the antibody or an antigen-binding fragment thereof of claim 1 and a pharmaceutically acceptable carrier.

38. The pharmaceutical composition according to claim 37, wherein the pharmaceutical composition is intended for the treatment or prevention of human metapneumovirus infection.

39. An isolated nucleic acid encoding the amino acid sequence of the antibody or an antigen-binding fragment thereof according to claim 1, an isolated nucleic acid encoding an amino acid sequence of any of SEQ ID NOs: 1, 6, 11, 16, 21, 26, 31, 36, 41, 46, 51, 56, 61, 66, 71, 76, 81, 86, 91, 96, 101, 106, 111, 116, 121, and 126, or an isolated nucleic acid hybridizing under high stringent conditions to any of these nucleic acids.

40. A recombinant expression vector comprising an isolated nucleic acid according to claim 39 incorporated therein.

41. A host cell comprising a recombinant expression vector according to claim 40 introduced thereinto.
